(19) United States
(12) Patent Application Publication
(10) Pub. No.: US 2026/0002103 A1
(43) Pub. Date: Jan. 1, 2026

(54) HYDROGEN AND ALGAL PROTEIN CO-PRODUCTION DEVICE

(71) Applicant: HENAN AGRICULTURAL UNIVERSITY, Zhengzhou (CN)

(72) Inventors: Zhiping ZHANG, Zhengzhou (CN); Yinggang JIAO, Zhengzhou (CN); Jinrui ZHANG, Zhengzhou (CN); Mingming CHEN, Zhengzhou (CN); Mingyang LIU, Zhengzhou (CN); Bing HU, Zhengzhou (CN); Shixiong LAN, Zhengzhou (CN); Yameng LI, Zhengzhou (CN); Hong LIU, Zhengzhou (CN); Zihan LIU, Zhengzhou (CN); Shuaikang XU, Zhengzhou (CN)

(73) Assignee: HENAN AGRICULTURAL UNIVERSITY, Zhengzhou (CN)

(21) Appl. No.: 19/032,004

(22) Filed: Jan. 18, 2025

(30) Foreign Application Priority Data
Jun. 26, 2024 (CN) 202410839769.6

(51) Int. Cl. C12M 1/00; C12M 1/06

(52) U.S. Cl. CPC C12M 21/02; C12M 23/38; C12M 27/02; C12M 31/10; C12M 39/00

(57) ABSTRACT

The present disclosure provides a hydrogen and algal protein co-production device, comprising: a hydrogen-producing substrate premix unit including a hydrogen-producing substrate premix box; a biological hydrogen-generating reactor including a hydrogen-producing substrate conveying assembly, a rotary power assembly, a hollow shaft, a hydrogen-generating reaction tube, a reaction tube top cover, an agitation release assembly, and an inner wall lighting assembly, wherein a top of the hollow shaft is connected to the hydrogen-producing substrate premix box, a top peripheral side of the hollow shaft is connected to the rotary power assembly, a bottom of the hollow shaft is connected to the agitation release assembly; and a hydrogen-producing tail liquid recovery unit being connected to the hydrogen-generating reaction tube and a hydrogen-producing tail liquid dilution unit, the hydrogen-producing tail liquid dilution unit being connected to a chlorella culture unit, and the chlorella culture unit being connected to a chlorella enrichment unit.

### CROSS-REFERENCE TO RELATED APPLICATIONS

The present disclosure claims priority to Chinese present disclosure No. 202410839769.6, filed Jun. 26, 2024, the entire contents of which are incorporated herein by reference.

### TECHNICAL FIELD

The present disclosure relates to the field of biological hydrogen production technology, specifically relates to a hydrogen and algal protein co-production device.

### BACKGROUND

At present, the gradual depletion of fossil energy makes more attention to the use of hydrogen energy, biological hydrogen production is the use of microorganisms to release hydrogen from their own metabolism, the hydrogen production conditions are mild, environmentally friendly and abundant sources of raw materials and is considered to be the main alternative form of hydrogen energy production in the future. In the comparison of various types of bio-hydrogen technology, photosynthetic bacteria hydrogen production not only has a high capacity for hydrogen production, but also can utilize a variety of organic waste as a raw material for hydrogen production, which can achieve the dual goals of hydrogen production and waste treatment and become a hot issue in the research of hydrogen technology. However, the hydrogen production tail liquid generated after the hydrogen production by photosynthetic bacteria is not easy to be treated.

Using Chlorella to remediate polluted water bodies can effectively reduce the nitrogen, phosphorus, and organic matter content of sewage, Chlorella is a kind of autotrophic unicellular algae with low growth conditions, using carbon dioxide and carbonate as the carbon source, the nitrogen in the environment as the nitrogen source, and inorganic phosphates as the phosphorus source, utilizing chlorophyll in algal cells for photoautotrophy has high photosynthetic efficiency, fast growth, and strong environmental adaptability. Chlorella is used to treat hydrogen-producing tailings.

Therefore, a hydrogen and algal protein co-production device is proposed, which ensures a continuous production of biohydrogen while adding additional harvested products, realizing the coupling co-production of “bacteria-hydrogen-algae-protein” coupling co-production, to achieve energy saving and emission reduction.

### SUMMARY

One or more embodiments of the present disclosure provide a hydrogen and algal protein co-production device, comprising: a hydrogen-producing substrate premix unit including a hydrogen-producing substrate premix box; a biological hydrogen-generating reactor including a hydrogen-producing substrate conveying assembly, a rotary power assembly, a hollow shaft, a hydrogen-generating reaction tube, a reaction tube top cover, an agitation release assembly, and an inner wall lighting assembly, wherein a top opening of the hydrogen-generating reaction tube is equipped with the reaction tube top cover, the reaction tube top cover is rotationally connected to the hollow shaft, a top of the hollow shaft is connected to the hydrogen-producing substrate premix box through the hydrogen-producing substrate conveying assembly, a top peripheral side of the hollow shaft is connected to the rotary power assembly, a bottom of the hollow shaft is connected to the agitation release assembly, and an inner wall of the hydrogen-generating reaction tube is arranged with the inner wall lighting assembly; and a hydrogen-producing tail liquid recovery unit being connected to the hydrogen-generating reaction tube, and the hydrogen-producing tail liquid recovery unit being connected to a hydrogen-producing tail liquid dilution unit, the hydrogen-producing tail liquid dilution unit being connected to a chlorella culture unit, and the chlorella culture unit being connected to a chlorella enrichment unit.

### BRIEF DESCRIPTION OF THE DRAWINGS

The present disclosure will be further illustrated by way of exemplary embodiments, which will be described in detail by means of the accompanying drawings. The embodiments are not limiting, and in the embodiments, the same numbering denotes the same structure, wherein:

FIG. 1 is a schematic diagram illustrating a structure of a hydrogen and algal protein co-production device according to some embodiments of the present disclosure;

FIG. 2 is a schematic diagram illustrating a partial structure of a hydrogen and algal protein co-production device according to some embodiments of the present disclosure;

FIG. 3 is a schematic diagram illustrating a structure of a biological hydrogen-generating reactor according to some embodiments of the present disclosure;

FIG. 4 is a schematic diagram illustrating a structure of a transparent hollow panel and a movable light according to some embodiments of the present disclosure;

FIG. 5 is a schematic diagram illustrating a structure of a hydrogen-producing tail liquid recovery unit according to some embodiments of the present disclosure;

FIG. 6 is a schematic diagram illustrating a partial structure of a hydrogen-producing tail liquid recovery unit according to some embodiments of the present disclosure;

FIG. 7 is a schematic diagram illustrating a partial structure of a hydrogen and algal protein co-production device according toother embodiments of the present disclosure;

FIG. 8 is a schematic diagram illustrating a structure of a chlorella culture unit according to some embodiments of the present disclosure;

FIG. 9 is a schematic diagram illustrating a partial structure of a hydrogen and algal protein co-production device according to some other embodiments of the present disclosure;

FIG. 10 is an enlarged structure schematic diagram at A in FIG. 9;

FIG. 11 is a schematic diagram illustrating a structure of a chlorella enrichment unit according to some embodiments of the present disclosure;

FIG. 12 is a schematic diagram illustrating a structure of a hydrogen-producing substrate premix unit according to some embodiments of the present disclosure;

FIG. 13 is a scenario diagram illustrating a processing unit according to some embodiments of the present disclosure;

FIG. 14 is a schematic diagram illustrating a determination model according to some embodiments of the present disclosure.

### DETAILED DESCRIPTION

In order to more clearly illustrate the technical solutions of the embodiments of the present disclosure, the following will briefly introduce the accompanying drawings that are required to be used in the description of the embodiments. Obviously, the accompanying drawings in the following description are only some examples or embodiments of the present disclosure. It is possible for a person of ordinary skill in the art to apply the present disclosure to other similar scenarios in accordance with these drawings without creative labor.

With the gradual depletion of fossil energy sources, hydrogen is attracting attention as an alternative energy source. The biological hydrogen-generating technology, which may utilize the metabolism of microorganisms to release hydrogen, is regarded as a major alternative form of hydrogen production in the future due to its mildness, environmental friendliness, and abundance of raw materials. The photosynthetic bacteria hydrogen production has become a research hotspot due to its efficient utilization of waste and hydrogen production, but its tailings are not easy to deal with. Therefore, some embodiments of the present disclosure provide a hydrogen and algal protein co-production device, which utilizes the characteristics that Chlorella vulgaris can efficiently purify the water body and has simple growth conditions to treat the tail liquid of the photosynthetic bacteria hydrogen production. The device ensures the continuous production of biological hydrogen, while also adds additional harvested products, thereby achieves the purpose of emission reduction, carbon reduction and energy saving.

In some embodiments, the hydrogen and algal protein co-production device includes a hydrogen-producing substrate premix unit 2, a biological hydrogen-generating reactor 3, and a hydrogen-producing tail liquid recovery unit 4.

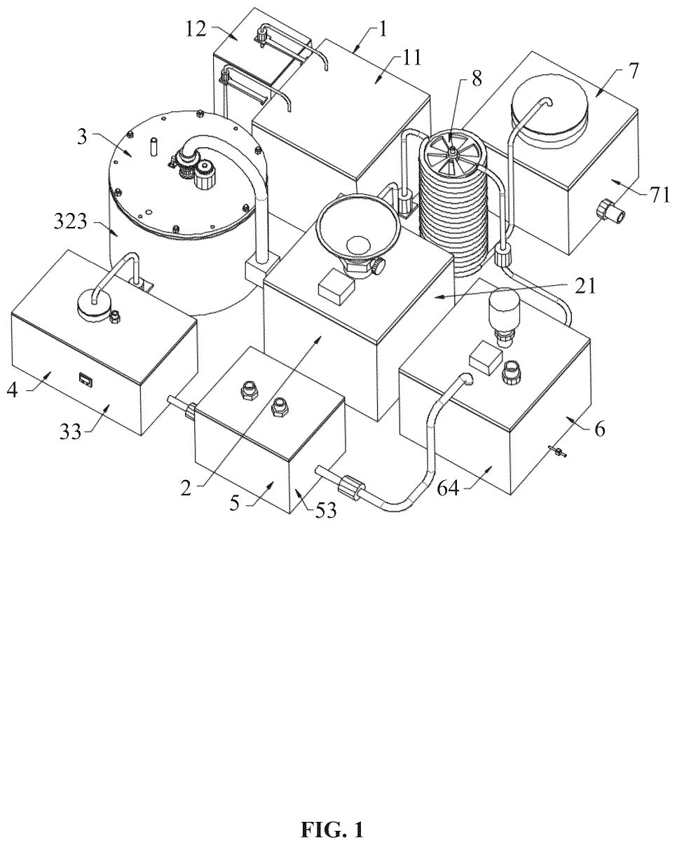

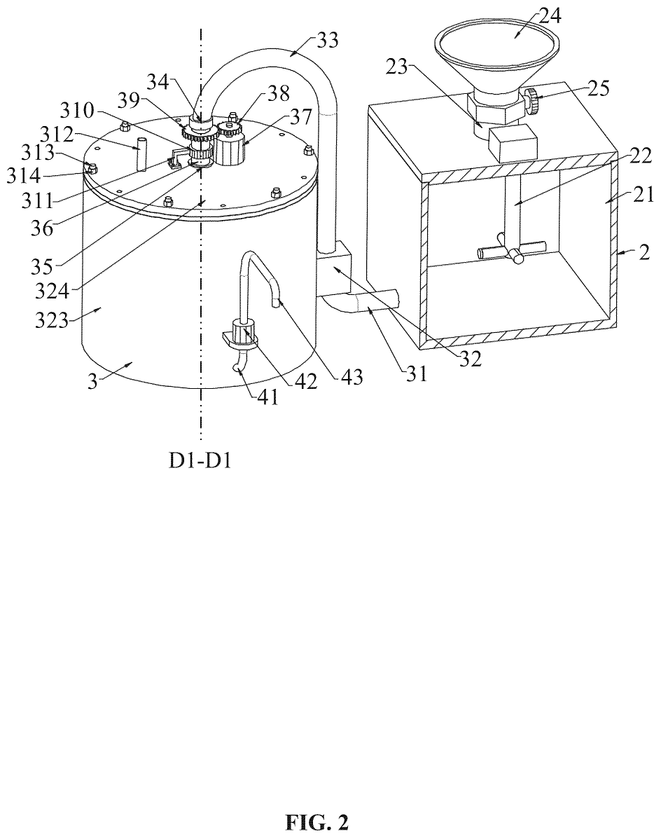

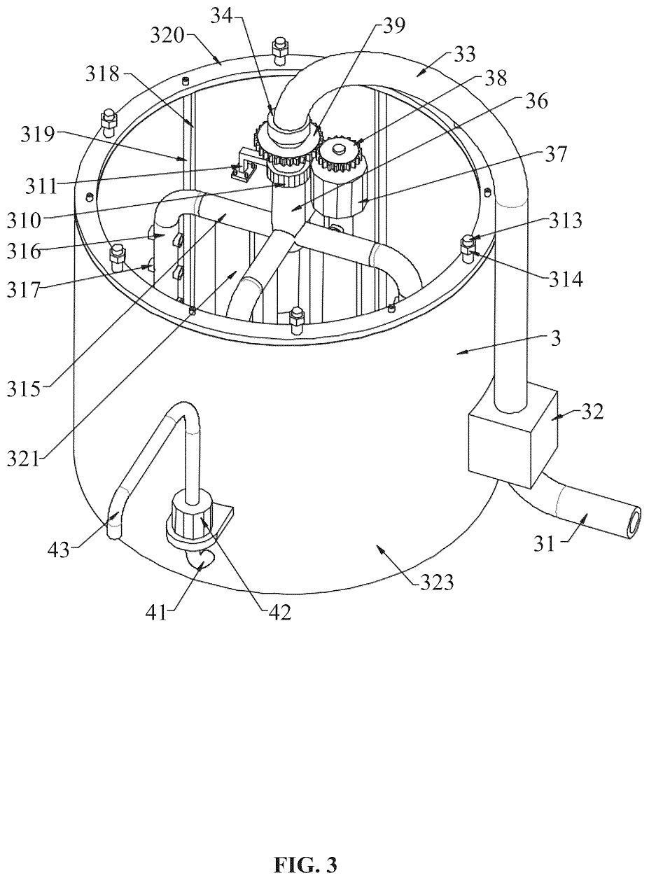

FIG. 1 is a schematic diagram illustrating a structure of a hydrogen and algal protein co-production device according to some embodiments of the present disclosure. FIG. 2 is a schematic diagram illustrating a partial structure of a hydrogen and algal protein co-production device according to some embodiments of the present disclosure. FIG. 3 is a schematic diagram illustrating a structure of a biological hydrogen-generating reactor according to some embodiments of the present disclosure.

In some embodiments, as shown in FIG. 1 and FIG. 3, the hydrogen-producing substrate premix unit 2 includes a hydrogen-producing substrate premix box 21; the biological hydrogen-generating reactor 3 includes a hydrogen-producing substrate conveying assembly, a rotary power assembly, a hollow shaft 36, a hydrogen-generating reaction tube 323, a reaction tube top cover 324, an agitation release assembly, and an inner wall lighting assembly, wherein a top opening of the hydrogen-generating reaction tube 323 is equipped with the reaction tube top cover 324, the reaction tube top cover 324 is rotationally connected to the hollow shaft 36, a top of the hollow shaft 36 is connected to the hydrogen-producing substrate premix box 21 through the hydrogen-producing substrate conveying assembly, a top peripheral side of the hollow shaft 36 is connected to the rotary power assembly, a bottom of the hollow shaft 36 is connected to the agitation release assembly, and an inner wall of the hydrogen-generating reaction tube 323 is arranged with the inner wall lighting assembly; and a hydrogen-producing tail liquid recovery unit 4 is connected to the hydrogen-generating reaction tube 323, and the hydrogen-producing tail liquid recovery unit 4 is connected to a hydrogen-producing tail liquid dilution unit 5, the hydrogen-producing tail liquid dilution unit 5 is connected to a chlorella culture unit 6, and the chlorella culture unit 6 is connected to a chlorella enrichment unit 7.

The hydrogen-producing substrate premix unit 2 is a unit for premixing hydrogen-producing substrates. The hydrogen-producing substrates include a straw waste and photosynthetic bacteria solution.

In some embodiments, as shown in FIG. 2, the hydrogen-producing substrate premix unit 2 includes a first mixing unit 22, a hydrogen-producing substrate addition pipe 23, a hopper 24, a first solenoid valve 25, and a top of the hydrogen-producing substrate premix box 21 is connected to the hopper 24 via the hydrogen-producing substrate addition pipe 23. The first solenoid valve 25 is mounted in a middle of the hydrogen-producing substrate addition tube 23, and the straw waste may be placed in the hopper 24. The first solenoid valve 25 is opened, the straw waste inside the hopper 24 enters into the hydrogen-producing substrate premix box 21 through the hydrogen-producing substrate addition pipe 23, and the photosynthetic bacteria solution may also be added to the hydrogen-producing substrate premix box 21 through the hopper 24. The hydrogen-producing substrate premix box 21 is installed with a first mixing unit 22, the first mixing unit 22 is used to mix the hydrogen-producing substrate, such as the straw waste, and the photosynthesizing bacteria. The first mixing unit 22 is also used to smash the straw waste. Exemplarily, the first mixing unit 22 includes a mixing motor, a rotating shaft, and a mixing paddle, and an output shaft of the mixing motor is connected to the rotating shaft, and the rotating shaft is connected to the mixing paddle.

The hydrogen-producing substrate conveying assembly is used to convey the mixture into the biological hydrogen-generating reactor 3. The mixture may be a mixture of the straw waste and the photosynthetic bacteria. In some embodiments, as shown in FIG. 2, the hydrogen-producing substrate conveying assembly includes a third delivery pipe 31, a third constant-flow pump 32, a fourth delivery pipe 33, and a swivel connector 34. A bottom side of the hydrogen-producing substrate premix box 21 is connected to one end of the third delivery pipe 31, and the other end of the third delivery pipe 31 is connected to the inlet of the third constant-flow pump 32. An outlet of the third constant-flow pump 32 is connected to one end of the fourth delivery pipe 33, and the other end of the fourth delivery pipe 33 is connected to the top of the hollow shaft 36 via the swivel connector 34. The swivel connector 34 is a closed swivel connector for conveying the media. The swivel connector 34 is capable of 360° rotation. While the hollow shaft 36 maintains a stable connection with the fourth delivery pipe 33 while rotating, the third constant-flow pump 32 pumps the mixture in the hydrogen-producing substrate premix box 21 through the third delivery pipe 31, and then pumps the mixture through the fourth delivery pipe 33 and rotary joint 34 into the hollow shaft 36.

The rotary power assembly is an assembly for providing rotational power to the hollow shaft 36. In some embodiments, as shown in FIG. 2-FIG. 3, the rotary power assembly includes an agitation motor 37, a first gear 38, and a second gear 39. The second gear 39 is fixedly connected to a top peripheral side of the hollow shaft 36. The reaction tube top cover 324 is fixedly connected with an agitation motor 37, and an output shaft of the agitation motor 37 is fixedly connected with a first gear 38. The first gear 38 is meshed with a second gear 39. When the agitation motor 37 is working, the hollow shaft 36 is driven to rotate by the transmission of the first gear 38 and the second gear 39.

In some embodiments, as shown in FIG. 2, the biological hydrogen-generating reactor 3 further includes a tapered roller bearing 35 and a middle portion of the reaction tube top cover 324 rotationally connected to a hollow shaft 36 via the tapered roller bearing 35.

In some embodiments, the middle of the reaction tube top cover 324 may be rotationally coupled to the hollow shaft 36 in a variety of ways. For example, a tapered roller bearing 35 may be mounted in the middle portion of the reaction tube top cover 324, and the hollow shaft 36 may pass through and rotate smoothly within the tapered roller bearing 35.

In some embodiments, the hollow shaft 36 may be placed vertically. Exemplarily, as shown in FIG. 2, the vertical direction may be an axis direction D1-D1 of the biological hydrogen-generating reactor 3.

The agitation release assembly is used to further agitate the mixture to fully mix the straw waste and photosynthetic bacteria. In some embodiments, as shown in FIG. 3, the agitation release assembly includes a plurality of horizontal pipes 315, a agitation vertical pipe 316, and a plurality of releasing ports 317, the plurality of horizontal pipes 315 are connected to an outside of a bottom end of the hollow shaft 36, one end of each of the plurality of horizontal pipes 315 away from the hollow shaft 36 is fixedly connected to a top end of the agitation vertical pipe 316, respectively, and the plurality of releasing ports 317 is provided at an outside of the agitation vertical pipe 316.

The horizontal pipe 315 is an assembly used to transport the mixture within the hollow shaft 36 to the agitation vertical pipe 316.

In some embodiments, the plurality of horizontal pipes 315 may be connected to the bottom peripheral side of the hollow shaft 36 in a variety of ways. For example, as shown in FIG. 3, four horizontal pipes 315 are connected to the bottom peripheral side of the hollow shaft 36 in an annular array.

In some embodiments, the plurality of releasing ports 317 are provided vertically equidistant from each other.

In some embodiments, as shown in FIG. 3, the plurality of releasing ports 317 are provided at equal distances along an axial direction of the agitation vertical pipe 316 on both sides of the stirring vertical pipe 316.

Exemplarily, four releasing ports 317 are provided on each side of the agitation vertical pipe 316, and the mixture entering the hollow shaft 36 enters the respective agitation vertical pipe 316 through the horizontal pipe 315, and then enters into the hydrogen-generating reaction tube 323 through the releasing ports 317. With the rotation of the hollow shaft 36, the mixture is evenly released, and through the agitation of the agitation vertical pipe 316, the straw waste and the photosynthetic bacteria in the mixture are evenly mixed, and the photosynthetic bacteria decompose and treat the straw wastes by the principle of photo fermentation, and generate hydrogen.

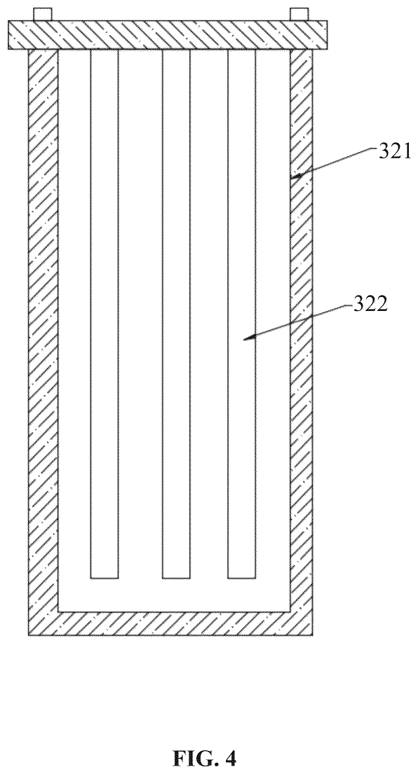

FIG. 4 is a schematic diagram illustrating a structure of a transparent hollow panel and a movable light according to some embodiments of the present disclosure.

In some embodiments, as shown in FIG. 2-FIG. 4, the biological hydrogen-generating reactor 3 further includes an electrical slip ring 310, a slip ring bracket 311, a gas collection pipe 312, a transparent hollow panel 321, and a movable light 322.

In some embodiments, the transparent hollow panel 321 is fixedly connected to the bottom of each horizontal pipe 315, respectively, and a plurality of movable lights 322 are provided at equal distances within each transparent hollow panel 321, respectively. Exemplarily, as shown in FIG. 4, the count of the movable lights 322 may be three.

In some embodiments, the electrical slip ring 310 is mounted on the top peripheral side of the hollow shaft 36, the electrical slip ring 310 is mounted to the reaction tube top cover 324 via the slip ring bracket 311, the electrical slip ring 310 is electrically connected to an external controller. The inner side of the electrical slip ring 310 is connected to a built-in insulating cable, and the built-in insulating cable passes through the hollow shaft 36 and the wire holes in the inner wall of the horizontal pipes 315 and is electrically connected to the movable light 322.

The movable light 322 rotates in synchronization with the hollow shaft 36, and the movable light 322 is powered by the electrical slip ring 310 and the built-in insulating cable, which can avoid the cable from causing kinking during rotation. The reaction tube top cover 324 is interspersed with a gas collection tube 312, and the gas containing hydrogen in the hydrogen-generating reaction tube 323 may be discharged and collected through the gas collection tube 312.

In some embodiments, as shown in FIG. 3, the inner wall lighting assembly includes a positioning mounting column 313, a limiting nut 314, a transparent baffle 318, a vertical light 319, and a light mounting ring 320.

The positioning mounting column 313 and the limiting nut 314 are used in conjunction to stabilize mounting of the light mounting ring 320 and the reaction tube top cover 324. The light mounting ring 320 is used to mount the vertical light 319.

In some embodiments, a plurality of troffers are provided in an annular array on an inner wall of the hydrogen-generating reaction tube 323. Exemplarily, the count of the troffers may be six or some other number.

In some embodiments, the transparent baffle 318 is provided on one side of each troffer proximate to a center of the hydrogen-generating reaction tube 323, respectively, and a plurality of positional mounting columns 313 are provided in an annular array on the top of the hydrogen-generating reaction tube 323. The plurality of positioning mounting column 313 may be vertically disposed. Exemplarily, the number of positioning mounting column 313 may be six.

In some embodiments, as shown in FIG. 3, the light mounting ring 320 is provided at the top of the hydrogen-generating reaction tube 323. A bottom portion of the light mounting ring 320 corresponding to the troffer is connected to a top portion of the vertical light 319, and the vertical light 319 is disposed within the corresponding troffer. The top portion of each positioning mounting column 313 passes through the locating mounting holes on the light mounting ring 320 and the reaction tube top cover 324 in turn, and is threaded with a limiting nut 314, and the reaction tube top cover 324 is provided with a wiring harness at a position corresponding to the vertical light 319.

Closing the side of the troffer by the transparent baffle 318 prevents the mixture from entering into the light tank and affecting the normal operation of the vertical light 319. The vertical light 319 may emit light when working, and the light through the transparent baffle 318 can act on the photosynthetic bacteria, allowing the photosynthetic bacteria to carry out a photo-fermentation operation to produce hydrogen.

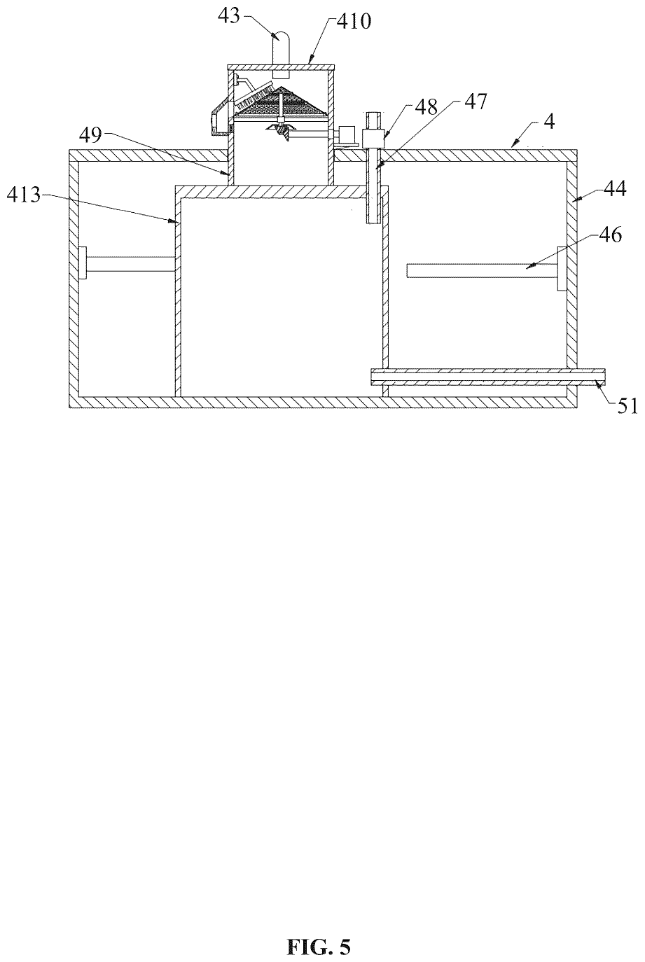

FIG. 5 is a schematic diagram illustrating a structure of a hydrogen-producing tail liquid recovery unit according to some embodiments of the present disclosure.

In some embodiments, as shown in FIG. 5, the hydrogen-producing tail liquid recovery unit 4 includes a heating water tank 44, a hydrogen-producing tail liquid conveying assembly, a solid-liquid separating mechanism, a sterilization assembly, and a sterilization box 413, the sterilization box 413 is provided inside the heating water tank 44, and the sterilization assembly is arranged on the heating water tank 44, the solid-liquid separating mechanism is connected to a top of the sterilization box 413, a top of the solid-liquid separating mechanism extends to a top outer side of the heating water tank 44, and the top of the solid-liquid separating mechanism is connected to the hydrogen-generating reaction tube 323 through the hydrogen-producing tail liquid conveying assembly.

The hydrogen-producing tail liquid is tail liquid left behind after the completion of the reaction in the hydrogen-generating reaction tube 323. The hydrogen-producing tail liquid conveying assembly is used to convey the hydrogen-producing tail liquid to the solid-liquid separating mechanism.

The heating water tank 44 is used to heat the hydrogen-producing tail liquid. The heating water tank 44 is filled with water during the working.

The sterilization box 413 is used to accommodate the hydrogen-producing tail liquid that is subject to a decontamination process. In some embodiments, after the photo-fermentation operation, the hydrogen-producing tail liquid is left behind in the hydrogen-generating reaction tube 323, and the hydrogen-producing tail liquid conveying assembly feeds the hydrogen-producing tail liquid into the solid-liquid separating mechanism to achieve solid-liquid separation, and the separated liquid enters the sterilization box 413, and the sterilization assembly is used to sterilize the liquid in the sterilization box 413.

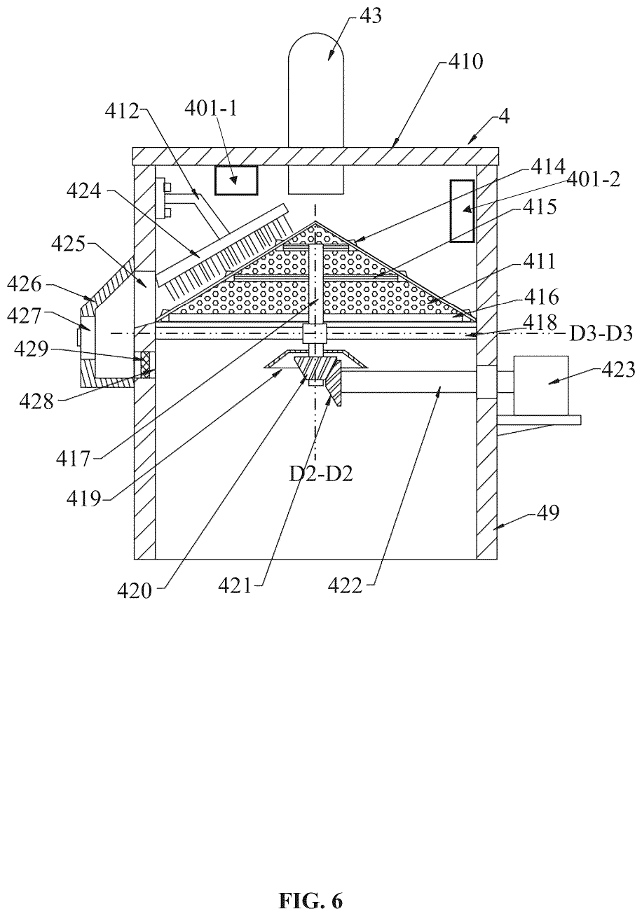

FIG. 6 is a schematic diagram illustrating a partial structure of a hydrogen-producing tail liquid recovery unit according to some embodiments of the present disclosure.

In some embodiments, as shown in FIG. 6, the solid-liquid separating mechanism includes a solid-liquid separating cylinder 49, a circular cover 410, a conical filter 411, a cleaning shaft 417, a shaft bracket 418, and a shaft-rotating power assembly, wherein a top of the sterilization box 413 is connected to the solid-liquid separating cylinder 49, and a top of the solid-liquid separating cylinder 49 is connected to a circular groove on a top of the heating water tank 44, the circular cover 410 is arranged on a top of the solid-liquid separating cylinder 49, the shaft bracket 418 is provided in a middle of the solid-liquid separating cylinder 49, the cleaning shaft 417 being rotationally connected in the middle of the shaft bracket 418, a bottom end of the cleaning shaft 417 is connected with the shaft-rotating power assembly, and a top of the cleaning shaft 417 is connected with an inside of the conical filter 411.

In some embodiments, the cleaning shaft 417 may be provided vertically, i.e., along a direction perpendicular to a horizontal plane.

The solid-liquid separating cylinder 49 is used to separate the solid and the liquid in the hydrogen-producing tail liquid.

In some embodiments, as shown in FIG. 6, a blocking ring 414 is provided at a top of the conical filter 411, wherein a diameter at the bottom of the conical filter 411 is slightly smaller than an inner diameter of the solid-liquid separating cylinder 49. Exemplarily, a plurality of blocking rings 414 are provided at the top of the conical filter 411.

The blocking ring 414 may block the flow of the hydrogen-producing tail liquid on the upper side of the conical filter 411. In some embodiments, the blocking ring 414 may allow liquid in the hydrogen-producing tail liquid to drop smoothly through the sieve holes in the conical filter 411 as it flows downward on the upper side of the conical filter 411, avoiding the hydrogen-producing tail liquid from rapidly pooling at the upper edge of the conical filter 411 causing slow filtration.

The hydrogen-producing tail liquid conveying assembly feeds the hydrogen-producing tail liquid into the solid-liquid separating cylinder 49, and the hydrogen-producing tail liquid flows to the top of the conical filter 411 and falls after the filtration of the conical filter 411. The hydrogen-producing tail liquid passes through the bottom side of the solid-liquid separating cylinder 49 and enters into the sterilization box 413, while the solid such as tail liquid residue in the hydrogen-producing tail liquid is retained on the top side of the conical filter 411. The shaft-rotating power assembly drives the cleaning shaft 417 to rotate relative to the shaft bracket 418, which in turn rotates the conical filter 411. The conical filter 411 may fling the tail liquid residue left on the upper side of the conical filter 411 to the upper edge of the conical filter 411 by centrifugal force, avoiding the tail liquid residue from blocking the sieve holes on the conical filter 411 on a wide scale affecting the filtering performance of the conical filter 411.

In some embodiments, a central axis of the cleaning shaft 417 is perpendicular to a central axis of the shaft bracket 418, and the cleaning shaft 417 rotates about its own central axis.

As shown in FIG. 6, a center axis D2-D2 of the cleaning shaft 417 is perpendicular to the center axis D1-D1 of the shaft bracket 418, and the cleaning shaft 417 rotates about its own center axis D2-D2.

In some embodiments, as shown in FIG. 6, the solid-liquid separating mechanism further includes an inner connecting rod 415, a reinforcing support ring 416, and a lower side of the conical filter 411 is provided with three reinforcing support rings 416. The conical filter 411 stably maintains a form with the help of the reinforcing support ring 416 and will not be easily deformed. The inner side of the two reinforcing support rings 416 on the upper side is connected to the cleaning shaft 417 through the inner connecting rods 415, and the rotation of the cleaning shaft 417 drives the inner connecting rods 415 and the reinforcing support rings 416 to rotate, thereby stably driving the conical filter 411 to rotate.

The shaft-rotating power assembly is used to provide rotational power to the cleaning shaft. In some embodiments, as shown in FIG. 6, the shaft-rotating power assembly includes a waterproof guard 419, a driven bevel gear 420, a drive bevel gear 421, a power shaft 422, and a power motor 423. In some embodiments, the shaft-rotating power assembly drives the cleaning shaft 417 to rotate with respect to the shaft bracket 418, which in turn spins the conical filter 411.

In some embodiments, the driven bevel gear 420 is fixedly coupled to the bottom end of the cleaning shaft 417, and the side of the solid-liquid separating cylinder 49 is rotationally coupled to the power shaft 422 via a sealed bearing.

The driven bevel gear 420 may transmit power by meshing the teeth of the driven bevel gear 420 with the teeth of the drive bevel gear 421.

In some embodiments, the power shaft 422 may be provided in a transverse direction. Wherein the transverse direction may be an axis direction D3-D3 of the shaft bracket 418.

In some embodiments, the power shaft 422 is fixedly connected to the drive bevel gear 421 at one end of the power shaft 422 disposed within the solid-liquid separating cylinder 49, the drive bevel gear 421 is meshed with the driven bevel gear 420, and the power shaft 422 is fixedly connected to the power motor 423 at one end of the power shaft 422 disposed at the outer side of the solid-liquid separating cylinder 49.

In some embodiments, the power motor 423 is fixed to an outer side of the solid-liquid separating cylinder 49 via a motor mount, and the power motor 423 is located on an upper side of the heating water tank 44, and the power motor 423 operates to drive the power shaft 422 to rotate. The cleaning shaft 417 is driven to rotate by the power shaft 422 through the transmission of the driven bevel gear 420 and the drive bevel gear 421.

In some embodiments, the cleaning shaft 417 is provided with a waterproof guard 419 located on the upper side of the driven bevel gear 420. The waterproof guard 419 may cover the upper side of the driven bevel gear 420 and the drive bevel gear 421, avoiding the liquid filtered by the conical filter 411 from falling directly onto the driven bevel gear 420 and the drive bevel gear 421, and keeping the driven bevel gear 420 and the drive bevel gear 421 working stably.

In some embodiments, as shown in FIG. 6, the solid-liquid separating mechanism also includes a cleaning brush 424, a tail liquid residue outlet 425, a tail liquid residue box 426, a tail liquid backflow port 428, and a backflow filter 429, the cleaning brush 424 is arranged on a top side of the solid-liquid separating cylinder 49, and bristles on a bottom side of the cleaning brush 424 are in contact with an upper surface of the conical filter 411, the tail liquid residue outlet 425 on a side of the solid-liquid separating cylinder 49 corresponds to a position of the cleaning brush 424, and a bottom of the tail liquid residue outlet 425 and a bottom of the conical filter 411 are arranged horizontally corresponding to each other, the tail liquid residue box 426 is fixedly connected to an outside of the solid-liquid separating cylinder 49, an inside of the tail liquid residue box communicates with the tail liquid residue outlet, a position of a bottom of the tail liquid residue box 426 corresponding to a side of the solid-liquid separating cylinder 49 is provided with the tail liquid backflow port 428, the tail liquid backflow port 428 is connected to an interior of the tail liquid residue box 426, and the backflow filter 429 is arranged in the tail liquid backflow port 428.

The tail liquid residue box 426 is used to collect tail liquid residue discharged from the tail liquid residue outlet 425.

In some embodiments, the tail liquid residue outlet 425 and the tail liquid residue box 426 are provided, which is convenient to clean up the tail liquid residue accumulated on the upper side of the conical filter 411 without opening the circular cover 410 each time the tail liquid residue is cleaned up, thus the solid-liquid separating mechanism is not interrupted, and the work efficiency is improved.

In some embodiments, the tail liquid residue box 426 is disposed on the upper side of the heating water tank 44, and the interior of the tail liquid residue box 426 communicates with the tail liquid residue outlet 425.

The tail liquid backflow port 428 may be used to return a portion of the tail liquid to the solid-liquid separating mechanism for reprocessing.

The backflow filter 429 may be used to filter the returned tail liquid.

If the tail liquid residue of the tail liquid residue box 426 still contains liquid, after filtration by the backflow filter 429, the liquid within the tail liquid residue is returned through the tail liquid backflow port 428 to the solid-liquid separating cylinder 49, which can improve the solid-liquid separation effect of the hydrogen-producing tail liquid.

In some embodiments, as shown in FIG. 6, the solid-liquid separating mechanism further includes a brush holder 412 and a residue box door 427. The solid-liquid separating cylinder 49 is connected to the brush holder 412 fixedly by screws on a top side inside the solid-liquid separating cylinder 49, and the brush holder 412 is connected to the cleaning brush 424. In some embodiments, the tail liquid residue box 426 is opened on a side away from the solid-liquid separating cylinder 49 with a residue cleanout opening, and the residue box door 427 is captured within the residue cleanout opening by a box door catch.

In some embodiments of the present disclosure, when the conical filter 411 is spinning, the tail liquid residue lefts on the upper side of the conical filter 411 is cleaned up by the cleaning brush 424, and falls into the tail liquid residue box 426 through the tail liquid residue outlet 425. When the tail liquid residue in the tail liquid residue box 426 is too much, the residue box door 427 is opened by the box door catch, the tail liquid residue in the tail liquid residue box 426 can be cleaned away through the residue cleaning port, and the cleaning does not affect the continuous operation of the solid-liquid separating mechanism.

The hydrogen-producing tail liquid conveying assembly is used to deliver the hydrogen-producing tail liquid to the solid-liquid separating mechanism. In some embodiments, as shown in FIG. 2 and FIG. 3, the hydrogen-producing tail liquid conveying assembly includes a fifth delivery pipe 41, a fourth constant-flow pump 42, and a sixth delivery pipe 43. The bottom side of the hydrogen-generating reaction tube 323 is connected to one end of the fifth delivery pipe 41, the other end of the fifth delivery pipe 41 is connected to an inlet of the fourth constant-flow pump 42, the outlet of the fourth constant-flow pump 42 is connected to one end of the sixth delivery pipe 43, and the other end of the sixth delivery pipe 43 passes through the middle of the circular cover 410.

In some embodiments, the fourth constant-flow pump 42 pumps the hydrogen-producing tail liquid within the hydrogen-generating reaction tube 323 through the fifth delivery pipe 41, and the hydrogen-producing tail liquid is sent into the solid-liquid separating cylinder 49 through the sixth delivery pipe 43 and is filtered through the cannular filter 411. The solids remain within the cannular filter 411, and the separated liquid falls into the sterilization box 413. The circular cover 410 is opened and the cannular filter 411 can be removed, thereby facilitating the cleaning of the solids left inside the cannular filter 411.

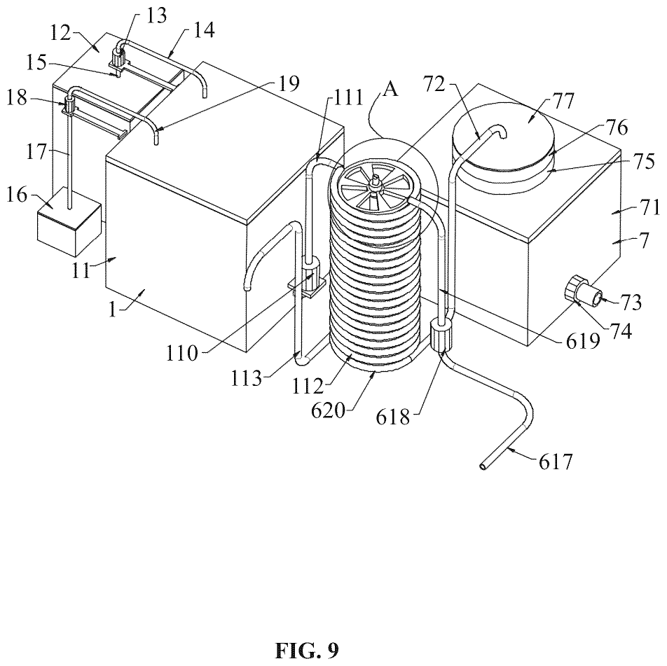

The sterilization assembly is used to decontaminate the hydrogen-producing tail liquid in the sterilization box. In some embodiments, as shown in FIG. 9, the sterilization assembly includes a temperature control switch 45, a heater 46, a first pH control pipe 47, and a first solenoid valve 48.

The first pH control pipe 47 is used to add acid-base solution to the hydrogen-producing tail liquid to adjust pH of the hydrogen-producing tail liquid. The first solenoid valve 48 is used to control the opening and closing of the first pH control pipe 47.

In some embodiments, as shown in FIG. 9, the heater 46 is mounted in the heating water tank 44, an input end of the heater 46 is electrically connected to an output end of the temperature control switch 45, the temperature control switch 45 is mounted on the side of the heating water tank 44, and a top end of the sterilization box 413 is connected to a bottom end of the first pH control pipe 47, a top end of the first pH control tube 47 passes through the top end of the heating water tank 44 and is mounted with the first solenoid valve 48.

In some embodiments of the present disclosure, the hydrogen-producing tail liquid in the sterilization box 413 is adjusted to be acidic (e.g., pH 4.5 to 5.5) by opening the first solenoid valve 48 and inputting acid solution into the sterilization box 413 via the first pH control tube 47 to facilitate sterilization of the hydrogen-producing tail liquid in the sterilization box 413. The temperature control switch 45 controls the heater 46 to heat the water within the heating water tank 44 to keep the water at a preset temperature to promote sterilization. For example, the water temperature within the heating water tank 44 is maintained at a temperature of 50° C. to 60° C., so that the hydrogen-producing tail liquid within the sterilization box 413 is maintained at 50° C. to 60° C. for ten minutes, to promote the sterilization effect on the sterilizing effect of the hydrogen-producing tail liquid within the sterilization box 413.

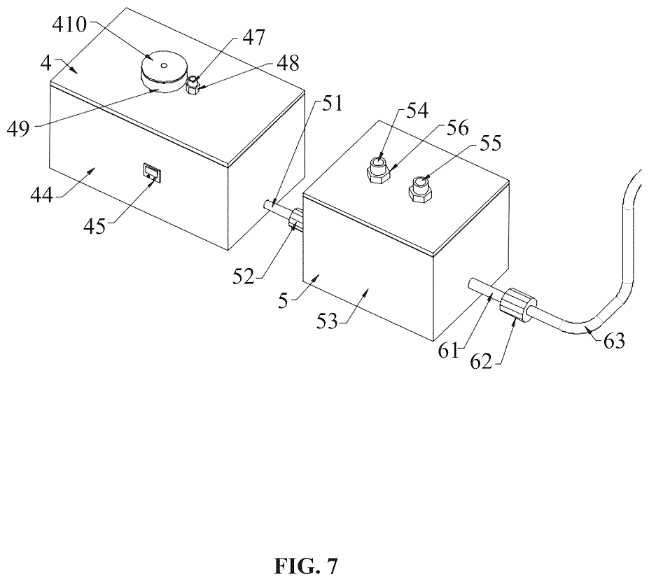

FIG. 7 is a schematic diagram illustrating a partial structure of a hydrogen and algal protein co-production device according to other embodiments of the present disclosure.

In some embodiments, as shown in FIG. 7, the hydrogen-producing tail liquid dilution unit 5 includes a first delivery pipe 51, a first constant-flow pump 52, a dilution box 53, and a dilution addition assembly.

In some embodiments, a bottom side of the sterilization box 413 is connected to one end of the first delivery pipe 51, the other end of the first delivery pipe 51 passes through a side of the heating water tank 44 and is connected to an inlet of the first constant-flow pump 52, an outlet of the first constant-flow pump 52 is connected to the dilution box 53, and the dilution box 53 is arranged with the dilution addition assembly.

The first delivery pipe 51 is used to deliver the hydrogen-producing tail liquid from the sterilization box 413 to the dilution box 53.

The first constant-flow pump 52 is used to provide a steady flow control for the first delivery pipe 51. In some embodiments, after the first constant-flow pump 52 feeds the decontaminated liquid in the sterilization box 413 through the first delivery pipe 51 to the dilution box 53, the dilution box 53 is added with water and nutrients by the dilution addition assembly to dilute the liquid separated from the original hydrogen-producing tail liquid.

In some embodiments, as shown in FIG. 7, the dilution addition assembly includes a water filling port 54, a nutrient filling port 55, and a second solenoid valve 56. The top of the dilution box 53 is provided with the water filling port 54 and the nutrient filling port 55, respectively, the water filling port 54 is used to refill the dilution box 53 with water, and the nutrient filling port 55 is used to add nutrient substance to the dilution box 53. The water filling port 54 and the nutrient substance filling port 55 are respectively provided with a second solenoid valve 56, and the second solenoid valve 56 is used for controlling an amount of water or nutrient substance to be added to the dilution box 53.

In some embodiments, as shown in FIG. 1, the chlorella culture unit 6 includes a diluent conveying assembly (also called a hydrogen-producing tail liquid diluent conveying assembly), a chlorella culture tank 64, an aeration assembly, a raw liquid addition assembly (also called a chlorella raw liquid addition assembly), and a chlorella lighting assembly, the dilution box 53 is connected to the chlorella culture tank 64 via the diluent conveying assembly, the chlorella culture tank 64 is provided with the aeration assembly and the chlorella lighting assembly, respectively, and the raw liquid addition assembly is arranged on a top of the chlorella culture tank 64.

In some embodiments, as shown in FIG. 7, the diluent conveying assembly includes an eighth delivery pipe 61, a sixth constant-flow pump 62, and a ninth delivery pipe 63, the bottom side of the dilution box 53 is connected to an end of the eighth delivery pipe 61, the other end of the eighth delivery pipe 61 is connected to an inlet of the sixth constant-flow pump 62, the outlet of the sixth constant-flow pump 62 is connected to one end of the ninth conveyor pipe 63, and the other end of the ninth delivery tube 63 is connected to the top of the chlorella culture tank 64, so that the diluent inside the dilution box 53 is fed into the chlorella culture tank 64 by the sixth constant-flow pump 62.

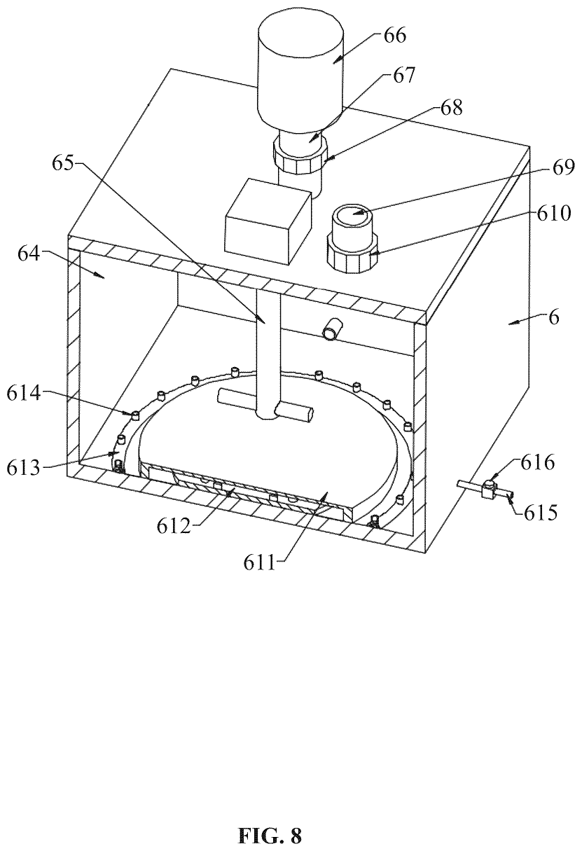

FIG. 8 is a schematic diagram illustrating a structure of a chlorella culture unit shown according to some embodiments of the present disclosure.

In some embodiments, as shown in FIG. 8, the aeration assembly includes an annular aeration pipe 613, an aeration head 614, an air supply pipe 615, and an air supply valve 616. The annular aeration pipe 613 is set at the bottom inside the chlorella culture tank 64, and the aeration head 614 is provided in a circular array on the annular aeration pipe 613. The annular aeration tube 613 is connected to one end of the air supply pipe 615, and the other end of the air supply pipe 615 extends to the outside of the chlorella culture tank 64. The air supply valve 616 is mounted at an outer end of the gas supply pipe 615, and the outer end of the gas supply pipe 615 is connected to an external gas source. The air supply pipe 615 is opened through the air supply valve 616, the external air source enters the annular aeration tube 613 through the air supply tube 615, and then aeration is carried out through the aeration head 614, so that the liquid in the chlorella culture tank 64 is in an oxygen-rich state and promoting the growth of chlorella.

In some embodiments, as shown in FIG. 8, the raw liquid addition assembly includes a chlorella raw liquid tank 66, a chlorella addition vertical pipe 67, and a third solenoid valve 68. The top of the chlorella culture tank 64 is connected to the chlorella raw liquid tank 66 via the chlorella addition vertical pipe 67. The third solenoid valve 68 is installed in the middle of the chlorella addition vertical pipe 67, and by opening the third solenoid valve 68, the chlorella raw liquid in the chlorella raw liquid tank 66 is added to the chlorella culture tank 64 through the chlorella addition vertical pipe 67, and chlorella grows rapidly in the chlorella culture tank 64, utilizing nutrients and substances in the hydrogen-producing tail liquid.

In some embodiments, as shown in FIG. 8, the chlorella lighting assembly includes a transparent round box 611 and a light board 612. The transparent round box 611 is mounted at the bottom inside the chlorella culture tank 64, and the light board 612 is mounted inside the transparent round box 611, and the light board 612 may take LED lights for lighting, the growth of the chlorella inside the chlorella culture tank 64 is promoted through the lighting of the light board 612. The second mixing unit 65 may stir the liquid inside the chlorella culture tank 64, so that the chlorella can fully receive the light from the light board 612.

In some embodiments, the chlorella culture unit 6 further includes a second pH control pipe 69, a fourth solenoid valve 610, and a second mixing unit 65. The second mixing unit 65 is mounted in the chlorella culture tank 64, the second pH control tube 69 is mounted on the top of the chlorella culture tank 64, and the fourth solenoid valve 610 is mounted on the second pH control pipe 69.

In some embodiments of the present disclosure, by opening the fourth solenoid valve 610, alkali solution is added to the chlorella culture tank 64 via the second pH control tube 69, so that the pH value of the liquid in the chlorella culture tank 64 is maintained at a preset range value, thereby promoting the growth of the chlorella. The second mixing unit 65 is used for fully mixing the alkali solution with the liquid in the chlorella culture tank 64. The preset range value may be 6.7-9.8.

In some embodiments of the present disclosure, the diluent conveying assembly delivers the diluent within the dilution box 53 into the chlorella culture tank 64, the raw liquid addition assembly adds chlorella to the chlorella culture tank 64, the chlorella lighting assembly provides light conditions to the chlorella culture tank 64, and the aeration assembly aerates at the bottom of the chlorella culture tank 64 to provide oxygen for the dilution liquid in the chlorella culture tank 64 to promote the rapid growth of the chlorella.

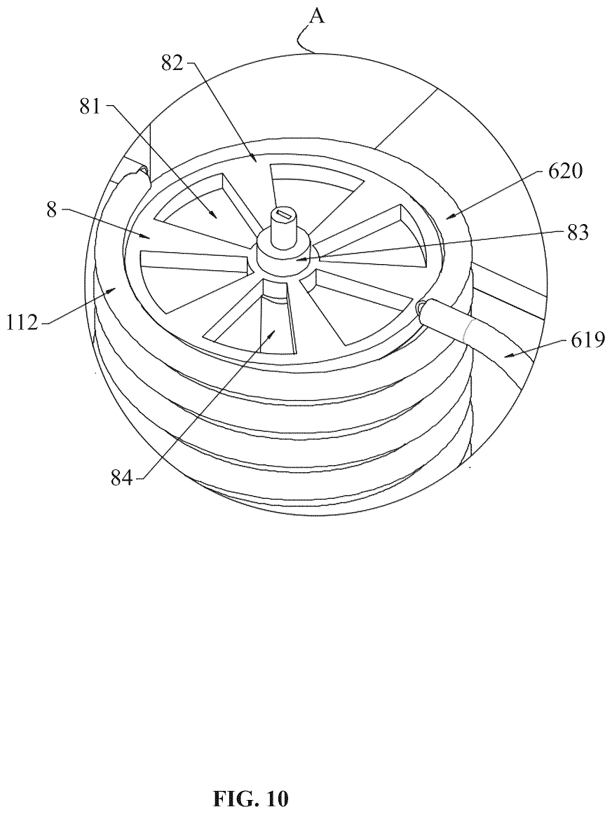

FIG. 9 is a schematic diagram illustrating a partial structure of a hydrogen and algal protein co-production device according to other embodiments of the present disclosure. FIG. 10 is an enlarged structure schematic diagram at A in FIG. 9.

In some embodiments, as shown in FIG. 9-FIG. 10, the chlorella culture unit 6 further includes a chlorella output assembly and a first spiral transparent pipe 620, the chlorella culture tank 64 is connected to a top end of the first spiral transparent pipe 620 via the chlorella output assembly, and the first spiral transparent pipe 620 is wrapped around a light mixing utilization unit 8.

The chlorella output assembly is used to output chlorella cultured to maturity in the chlorella culture tank 64. In some embodiments, as shown in FIG. 9, the chlorella output assembly includes a culture fluid delivery pipeline 617, a seventh constant-flow pump 618, and a culture fluid infusion pipeline 619. The bottom side of the chlorella culture tank 64 is connected to one end of the culture fluid delivery pipeline 617, and the other end of the culture fluid delivery pipeline 617 is connected to an inlet of the seventh constant-flow pump 618. The outlet of the seventh constant-flow pump 618 is connected to one end of the culture fluid delivery pipeline 619, and the other end of the culture fluid infusion pipeline 619 is connected to the tip of the first spiral transparent pipe 620. The seventh constant-flow pump 618 is used to pump away the liquid and chlorella inside the chlorella culture tank 64 and feed the liquid and chlorella into the first spiral transparent pipe 620.

In some embodiments, as shown in FIG. 10, the light mixing utilization unit 8 includes a transparent cylinder 81, a cylinder cover 82, a lamp holder 83, and a center illumination lamp 84. A top of the transparent cylinder 81 is fitted with the cylinder cover 82, a center of the lamp holder 83 is fitted with the cylinder cover 82, and the center illumination lamp 84 is fitted at the bottom of the lamp holder 83. The center illumination lamp 84 is disposed in a middle of the transparent cylinder 81, and the first spiral transparent pipe 620 is wrapped around an outer peripheral side of the transparent cylinder 81. The first spiral transparent pipe 620 may also be disposed in an inner side of the transparent cylinder 81. The cylinder cover 82 and the lamp holder 83 are cooperatively used for installing a center illumination lamp 84 in the middle of the transparent cylinder 81, and the light emitted from the center illumination lamp 84 may act on the flow of the chlorella in the first spiral transparent pipe 620, allowing the chlorella to continue to grow as passing through the first spiral transparent pipe 620.

In some embodiments of the present disclosure, the chlorella and the liquid inside the chlorella culture tank 64 are fed into the first spiral transparent pipe 620 by the chlorella output assembly, and the light mixing utilization unit 8 provides the chlorella flowing through the first spiral transparent pipe 620 with light to further promote the growth of the chlorella.

The chlorella enrichment unit is used to enrich chlorella from the waste liquid. In some embodiments, as shown in FIG. 9, the chlorella enrichment unit 7 includes a waste liquid inflow recovery chamber 71, a waste liquid delivery pipe 72, and an enrichment assembly, a top of the waste liquid inflow recovery chamber 71 is arranged with the enrichment assembly, and the enrichment assembly is connected to a bottom of the first spiral transparent pipe 620 via the waste liquid delivery pipe 72.

In some embodiments, excess chlorella and liquid within the first spiral transparent pipe 620 passes through the waste liquid delivery pipe 72 into the enrichment assembly to be filtered and enriched, and the remaining waste fluid enters the waste liquid inflow recovery chamber 71 to be recycled.

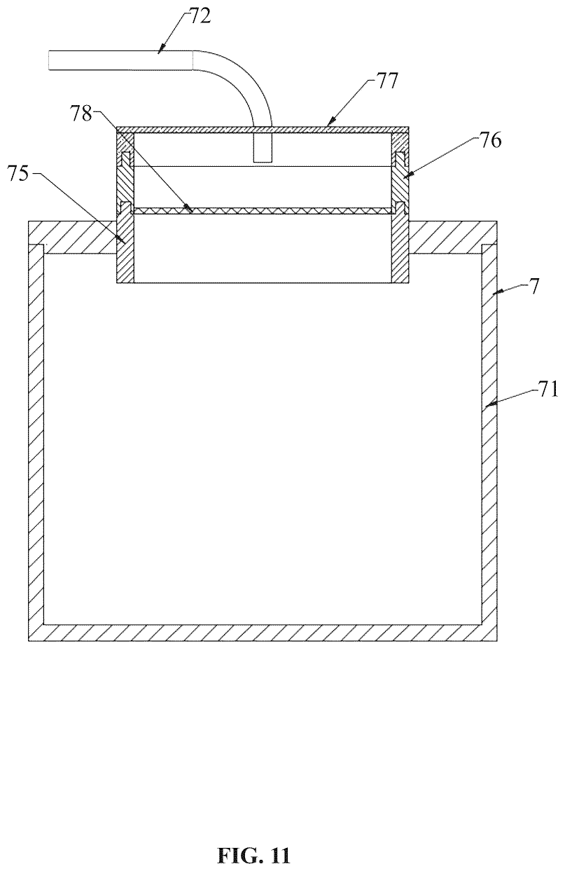

FIG. 11 is a schematic diagram illustrating a structure of a chlorella enrichment unit according to some embodiments of the present disclosure.

In some embodiments, as shown in FIG. 11, the enrichment assembly includes an enrichment mounting sleeve 75, a dismantling sleeve 76, an enrichment round cover 77, and an enrichment filter 78. The enrichment mounting sleeve 75 is interspersed at a top of the waste liquid inflow recovery chamber 71, a top of the enrichment mounting sleeve 75 is snapped with a bottom of the dismantling sleeve 76. The enrichment filter 78 is provided at a bottom of the dismantling sleeve 76, a top end of the dismantling sleeve 76 is snapped with a bottom of the enrichment round cover 77, and a middle portion of the enrichment round cover 77 is connected to the waste liquid delivery pipe 72.

In some embodiments, the chlorella and the liquid are delivered by the waste liquid delivery pipe 72 to the enrichment round cover 77, the chlorella is filtered and enriched by the enrichment filter 78 within the dismantling sleeve 76, and the liquid falls into the waste liquid inflow recovery chamber 71 to be recovered.

In some embodiments, a first snap ring is provided at the top of the enrichment mounting sleeve 75, and the first snap ring snaps to an annular first slot at the bottom of the dismantling sleeve 76. A second snap ring is provided at the top of the dismantling sleeve 76, and the second snap ring snaps to an annular second slot at the bottom of the enrichment round cover 77. The dismantling sleeve 76 can thus be disassembled and removed, so that the chlorella filtered off the enrichment filter 78 can be collected in a centralized manner.

In some embodiments, the chlorella enrichment unit 7 further includes a discharge pipeline 73 and a discharge valve 74. The bottom side of the waste liquid inflow recovery chamber 71 is connected to the discharge pipeline 73, and the discharge valve 74 is installed on the discharge pipeline 73, and by opening the discharge valve 74, the waste liquid inflow into the waste liquid inflow recovery chamber 71 may be discharged through the discharge pipeline 73.

In some embodiments, the chlorella within the chlorella culture unit 6 grows in the hydrogen-producing tailing dilution and eventually enters into the chlorella enrichment unit 7, and the chlorella enrichment unit 7 collects the chlorella. The chlorella can be used as a good animal feed due to the high protein content. The chlorella has advantages of low cost, low energy consumption, high efficiency, high yield, and high dissolved oxygen content in the effluent water, which allows treating the hydrogen-producing tail liquid with low cost and high efficiency.

In some embodiments, as shown in FIG. 1 and FIG. 9, the hydrogen and algal protein co-production device further includes a photosynthetic bacteria culture unit 1. The photosynthetic bacteria culture unit 1 includes a feed premixing box 11, a photosynthetic bacteria premix conveying assembly, a second spiral transparent pipe 112, and a second delivery pipe 113, and the feed premixing box 11 is connected to the top end of the second spiral transparent pipe 112 via the photosynthetic bacteria premix conveying assembly.

In some embodiments, the second spiral transparent pipe 112 may be provided in a variety of ways. For example, the second spiral transparent pipe 112 may be wrapped around an outer peripheral side of the transparent cylinder 81 in the light mixing utilization unit 8. As another example, the second spiral transparent pipe 112 may be provided on the inner side of the transparent cylinder 81.

In some embodiments, the light emitted from the center illumination lamp 84 through the transparent cylinder 81 and the second spiral transparent pipe 112 promotes the growth of photosynthetic bacteria within the second spiral transparent pipe 112, and the bottom end of the second spiral transparent pipe 112 is connected to the hydrogen-producing substrate premix box 21 via a second delivery pipe 113.

The feed premixing box 11 premixes the culture liquid of the photosynthetic bacteria and the raw liquid of the photosynthetic bacteria. In some embodiments, as shown in FIG. 9, the liquid in the feed premixing box 11 may be fed through the photosynthetic bacteria premix conveying assembly into the second spiral transparent pipe 112, and the photosynthetic bacteria in the second spiral transparent pipe 112 are subjected to light growth and multiplication by the light from the light mixing utilization unit 8. Then the photosynthetic bacteria are fed into the hydrogen-producing substrate premix box 21 through the second delivery pipe 113, where the photosynthetic bacteria are mixed with the straw waste in the hydrogen-producing substrate premix box 21.

In some embodiments, as shown in FIG. 9, the photosynthetic bacteria premix conveying assembly includes an eighth constant-flow pump 110 and a seventh delivery pipe 111. The bottom side of the feed premixing box 11 is connected to an inlet of the eighth constant-flow pump 110, the outlet of the eighth constant-flow pump 110 is connected to one end of the seventh delivery pipe 111, and the other end of the seventh delivery pipe 111 is connected to the top of the second spiral transparent pipe 112.

The eighth constant-flow pump 110 and the seventh delivery pipe 111 may be used to feed a mixture of the culture liquid and the photosynthetic bacteria raw liquid into the second spiral transparent pipe 112.

In some embodiments, as shown in FIG. 2, the photosynthetic bacteria culture unit 1 further includes a culture liquid storage tank 12, a fifth constant-flow pump 13, a culture liquid delivery pipe 14, a culture liquid pumping pipe 15, a photosynthetic bacteria raw liquid tank 16, a photosynthetic bacteria raw liquid pumping pipe 17, a second constant-flow pump 18, and a photosynthetic bacteria raw liquid delivery pipe 19.

In some embodiments, a top of the feed premixing box 11 is connected to one end of the culture liquid delivery pipe 14, the other end of the culture liquid delivery pipe 14 is connected to an outlet of the fifth constant-flow pump 13. An inlet of the fifth constant-flow pump 13 is connected to one end of the culture liquid pumping pipe 15, the other end of the culture liquid pumping pipe 15 extends through a top of the culture liquid storage tank 12 to a bottom within the culture liquid storage tank 12, and the culture liquid storage tank 12 contains a culture liquid of photosynthetic bacteria.

In some embodiments, the top of the feed premixing box 11 is connected to one end of the photosynthetic bacteria raw liquid delivery pipe 19, and the other end of the photosynthetic bacteria raw liquid delivery pipe 19 is connected to an outlet of the second constant-flow pump 18. An inlet of the second constant-flow pump 18 is connected to one end of the photosynthetic bacteria raw liquid pumping pipe 17, and the other end of the photosynthetic bacteria raw liquid pumping pipe 17 extends through a top of the photosynthetic bacteria raw liquid tank 16 and into a bottom of the photosynthetic bacteria raw liquid tank 16, and the photosynthetic bacteria raw liquid is contained in the photosynthetic bacteria raw liquid tank 16.

Notably, the first constant-flow pump 52, the second constant-flow pump 18, the third constant-flow pump 32, the fourth constant-flow pump 42, the fifth constant-flow pump 13, the sixth constant-flow pump 62, the seventh constant-flow pump 618, the eighth constant-flow pump 110 disclosed in the above embodiment also play the role of a throttle during the entire system operation to prevent backflow of liquid, a feed rate and a backflow rate may also be realized by the respective rotational speed, and the above power-using devices are all controlled to work by an external controller, and the control method adopts the prior art.

It should be noted that in the document, relational terms such as first and second, etc., are used only to distinguish one entity or operation from another and do not necessarily require or imply that any such actual relationship or order between those entities or operations is not necessarily required or implied. The terms “including,” “comprising,” or any other variant thereof, are intended to cover non-exclusive the terms “including,” “comprising,” or any other variant thereof, are intended to cover non-exclusive inclusion, such that a process, method, article, or apparatus comprising a set of elements includes not only those elements, but also other elements that are not explicitly listed, or that are inherent to such process, method, article, or apparatus.

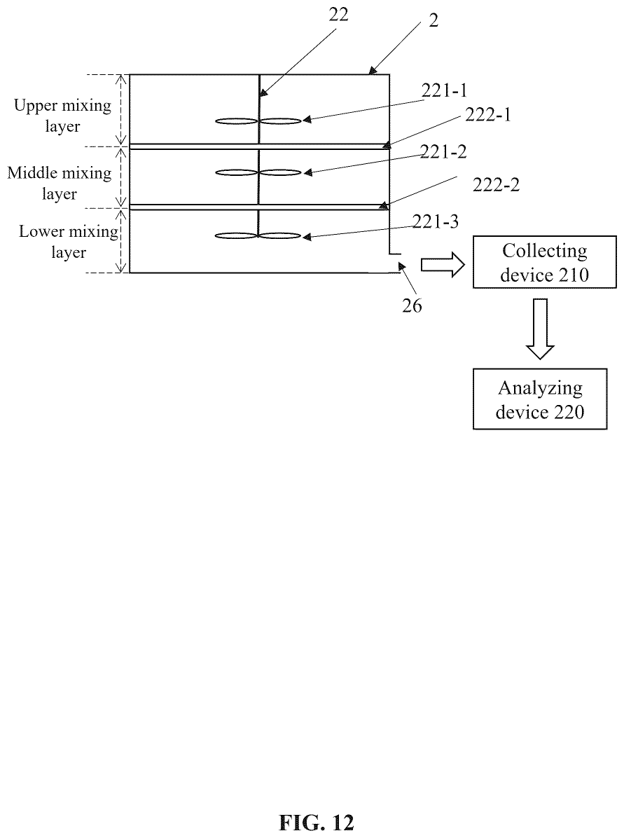

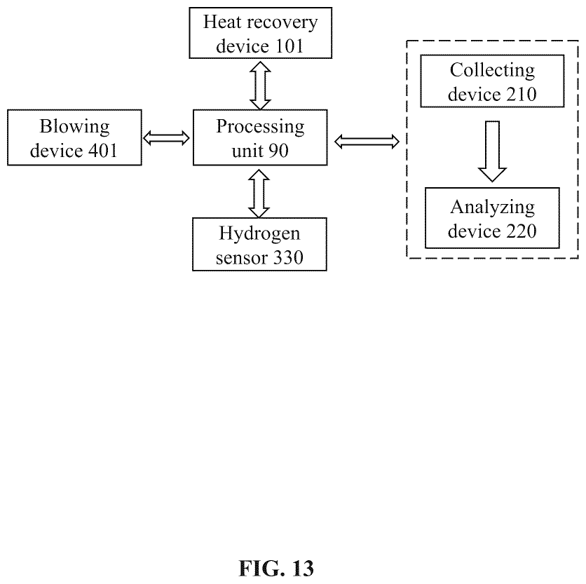

FIG. 12 is a schematic diagram illustrating a structure of a hydrogen-producing substrate premix unit according to some embodiments of the present disclosure. FIG. 13 is a scenario diagram illustrating a processing unit according to some embodiments of the present disclosure.

In some embodiments, the hydrogen and algal protein co-production device further includes a processing unit 90, the hydrogen-producing substrate premix unit 2 includes a first mixing unit 22, and the first mixing unit 22 includes a multi-layer mixer. A baffle is provided between each of the multi-layer mixer. The processing unit 90 is configured to: determine a mixing parameter of the first mixing unit based on straw initial feature.

More descriptions of the hydrogen-producing substrate premix unit 2 and the first mixing unit 22 may be found in FIG. 1-FIG. 11 and related descriptions.

The multi-layer mixer may include two or more mixing layers. The baffle is used to separate the different mixing layers, and the grid is opened when the mixing of the previous layer is complete, allowing the mixture to fall into the next layer. The different mixing layers may be separately provided with mixing blades, and the number and size of the mixing blades of the different mixing layers may be the same or different.

For example, as shown in FIG. 12, the multi-layer mixer includes an upper mixer 221-1, a middle mixer 221-2, and a lower mixer 221-3. A first baffle 222-1 is provided between the upper mixer 221-1 and the middle mixer 221-2, and a second baffle 222-2 is provided between the middle mixer 221-2 and the lower mixer 221-3. The first baffle 222-1 and the second baffle 222-2 divide the mixing space in which the first mixing unit 22 is located into an upper, middle, and lower layer. The upper layer is used for the initial agitation mixing. The middle layer is used for the main mixing. The lower layer carries out the final mixing and discharges the stirred mix.

In some embodiments, the multi-layer mixer may further include 2, 4, or other number of mixing layers.

The mixture is a mixture of the straw waste and the photosynthetic bacteria.

The processing unit 90 may be a processor or a server. For example, the processing unit 90 may include a combination of one or more of a microcontroller (MCU), an embedded processor, a graphics processing unit (GPU), or the like.

In some embodiments, the processing unit 90 may be mounted in or remotely communicatively coupled to the hydrogen and algal protein co-production device.

The straw initial feature includes a particle size, particle distribution, a volume or a weight, a moisture content, a temperature of a straw prior to mixing, or the like. The straw initial feature may be obtained by sensors or experimental measurements in advance, and automatically or manually entered into the processing unit 90.

The mixing parameter includes a mixing parameter of the multiple mixing layers. The mixing parameter for each layer includes a blade rotation speed of the mixer, the blade direction, or the like.

In some embodiments, the processing unit may determine the mixing parameter based on the straw initial feature by a variety of manners.

For example, the processing unit may look up s first preset table to determine the mixing parameter based on the straw initial feature. The first preset table represents a mapping relationship between the straw initial feature and the mixing parameter, and the mapping relationship may be set manually based on experience. For example, the blade rotational speed is positively correlated with the particle size, the volume or the weight, and the moisture content of the straw. As another example, the mixing process leads to an increase in temperature, and too high a temperature may lead to the inactivation of photosynthetic bacteria, an early reaction, or the like, and so the blade rotational speed may be set to be negatively correlated with the temperature.

Setting up the multi-layer mixer can make the mixing more uniform, and the straw waste and photosynthetic bacteria are fully mixed. At the same time, each layer of the mixer blades of the multi-layer mixer can have a cutting function, which can further chop the straw waste during the mixing process, accelerating the mixing of the straw and the photosynthesizing bacteria as well as the fermentation reaction. The mixing parameter is determined by the straw initial feature, which can improve the mixing efficiency for different scenes and different forms of straw.

In some embodiments, the processing unit is further configured to determine mixing target data based on straw types and moisture content; determine an upper mixing parameter based on the straw initial feature and a photosynthetic bacteria feature; determine an initial mixing feature based on the upper mixing parameter, a upper mixer feature, the straw initial feature, the photosynthetic bacteria feature, and an initial mixing degree; and determine a middle mixing parameter based on the initial mixing feature, a middle mixer feature, the mixing target data, and straw toughness.

The mixing target data includes a target particle size after mixing, a target mixing degree, or the like. The target particle size is a range in which the average size of the straw particles after mixing falls. The mixing target data includes middle target data and lower target data. The middle target data and the lower target data represent the mixing target data of the middle mixing layer and the lower mixing layer after mixing, respectively.

In some embodiments, the processing unit may query a second preset table to determine a straw toughness and a straw reactivity based on the straw types and the moisture content; and determine the mixing target data based on the straw toughness and the straw reactivity. The straw types may include wheat straw, corn stover, or other types. The processing unit may obtain the straw types uploaded by the user in a user terminal. The moisture content is a moisture content in the straw, which may be measured by a sensor or an experiment and automatically or manually input into the processing unit 90.

The straw toughness represents a degree of resistance of straw to crushing. The straw reactivity represents a vigor degree of the reaction of the straw in the presence of enzymes. The straw toughness and straw reactivity may be obtained by experimenting with different moisture contents and different types of straws and deposited in the second preset table.

The target particle size is negatively correlated with the straw toughness and positively correlated with the straw reactivity. The target mixing degree is positively correlated with the straw toughness and negatively correlated with the straw reactivity.

The photosynthetic bacteria feature includes a type, a concentration, and a volume of the photosynthetic bacteria. The photosynthetic bacteria feature may be uploaded for the user, or may be obtained from sensors or experimental measurements.

The upper mixing parameter include a blade speed and a blade direction of the upper mixer.

In some embodiments, the processing unit looks up a third preset table to determine the upper mixing parameter based on the straw initial feature and the photosynthetic bacteria feature. The third preset table includes a mapping relationship between the straw initial feature and the photosynthetic bacteria feature and the upper mixing parameter. The third preset table may be set empirically. For example, the rotational speed of the blades of the upper mixer is positively correlated with the particle size, the volume, or the weight, the moisture content, the concentration of the straw, and the volume of the photosynthetic bacteria.

The initial mixing feature is a mixing feature of the mixture after being stirred by the upper mixer. The mixing feature includes the particle size of the straw, the particle distribution, the weight/volume, the moisture content, the temperature, and the degree of mixing of the mixture.

The upper mixer feature includes the number of blades in the upper mixer and more. The upper mixer feature can be uploaded for the user.

The initial mixing degree is a mixing degree of the mixture (the straw waste and the photosynthetic bacteria) before mixing through the upper mixer. The initial mixing degree may be user uploaded or preset. For example, the initial mixing degree may be set to 0.

In some embodiments, the processing unit may determine the initial mixing feature based on the upper mixing parameter, the upper mixer feature, the straw initial feature, the photosynthetic bacteria feature, and the initial mixing degree, by a first prediction model.

The first prediction model is a machine learning model for prediction of the initial mixing feature. For example, the first prediction model may be a Neural Networks (NN) or other models. Inputs of the first prediction model include the upper mixing parameter, the upper mixer feature, the straw initial feature, the photosynthetic bacteria feature, and the initial mixing degree, and outputs include the initial mixing feature.

In some embodiments, the first prediction model may be obtained by training with a large number of first training samples and first labels corresponding to the first training samples. The processing unit may perform multiple rounds of iterations based on the large number of the first training samples and training with the first labels, and when an end-of-iteration condition is satisfied, the iteration is ended, and a trained first prediction model is obtained. The at least one round of iteration comprises: selecting one or more first training samples to be input into the initial first prediction model, obtaining a model prediction output corresponding to the one or more first training samples; and based on the model prediction output and the first labels corresponding to the first training samples, substituting into a formula for a predefined loss function, calculating a value of the loss function; according to the value of the loss function, inversely updating the model parameter in the initial first prediction model by a gradient descent method or other methods.

Each group of the training samples in the first training samples may include a sample upper mixing parameter, a sample upper mixer feature, a sample straw initial feature, a sample photosynthetic bacteria feature, and a sample initial mixing degree. The first training samples may be obtained based on historical data. The first labels may be sample initial mixing feature corresponding to the first training samples actually acquired after the subsequent mixing is completed. For example, the actual acquisition is performed by the analyzing device 220 (see FIG. 12).

In some embodiments, the inputs of the first prediction model further include the straw toughness. The first training sample also includes a sample straw toughness.

The middle mixer feature includes the number of blades in the middle mixer or the like. The middle mixing feature may be uploaded for the user.

The middle mixing parameter include a blade speed and blade direction of the middle mixer.

In some embodiments, the processing unit may construct a mixing feature vector based on the initial mixing feature, the middle target data, the middle mixer feature, and the straw toughness; based on the mixing feature vector, look up the first vector database and match to determine the middle mixing parameter. The first vector database includes a plurality of sets of reference mixing feature vectors and a corresponding reference middle mixing parameter. The matching method may comprise: finding a reference mixing feature vector in the first vector database that has a highest similarity to the mixing characteristic vector, and using the corresponding reference middle mixing parameter as the target middle mixing parameter. The similarity is negatively correlated with the vector distance. If there are multiple reference mixing characteristic vectors with the highest similarity, the target value is averaged.

The first vector database may be a Milvus Vector Database (Milvus) or Facebook AI Similarity Search (Faiss). The first vector database may be constructed based on experiment. For example, the straw is subjected to a mixing experiment, and the initial mixing feature, the straw toughness, and middle mixer feature are obtained as the reference initial mixing feature, the reference straw toughness, and the reference middle mixer feature; and the experimental middle mixing parameter is obtained as the reference mixing parameter. The straw data after mixing (e.g., particle size, degree of mixing, etc.) is recorded at the completion of experiment as the reference middle target data. The processing unit may obtain the reference initial mixing feature, the parametric middle target data, the reference firmness, and the reference mixer feature of the plurality of experiments to construct a plurality of reference mixing feature vectors, and construct a plurality of reference mixing feature vectors based on the plurality of reference mixing feature vectors and the corresponding reference mixing parameters to construct a first vector database.

In some embodiments, a direction of blades of the middle mixer may be in an opposite direction of blades of the upper mixer.

Based on the different features of the straw and photosynthetic bacteria and the different parameters of the mixer and the desired mixing effect, different upper mixing parameters and middle mixing parameters are determined, which can ultimately enable the upper mixing layer and middle mixing layer to achieve different mixing effects. At the same time, it can make preliminary mixing before the mixture entering into the lower mixing layer, reduce the mixing pressure of the lower mixing layer, and thus improve the mixing efficiency.

In some embodiments, the hydrogen and algal protein co-production device further includes a collecting device 210 and an analyzing device 220; and a mixture sampling port 26 is arranged with a bottom of the hydrogen-producing substrate premix unit 2. The collecting device 210 and the analyzing device 220 are communicatively coupled to the processing unit 90.

The collecting device 210 is used to collect the mixture from the mixture sampling port. For example, the collecting device 210 may be provided as a robotic arm.

The analyzing device 220 is used to analyze data from the mixture collected by the collecting device 210. For example, the analyzing device may be at least one of a laser particle size meter, a moisture meter, or a temperature probe.

In some embodiments, the processing unit is further configured to: control the collecting device 210 to obtain a first mixture from the mixture sampling port 26 and feed the first mixture to the analyzing device 220 for analysis to obtain mix sampling data; determine a middle mixing feature based on the straw toughness, the middle mixing parameter, the middle mixer feature, the initial mixing feature, and the photosynthetic bacteria feature; determine an actual mixing feature based on the middle mixing feature and the mix sampling data; determine a lower mixing parameter based on the actual mixing feature, a lower mixer feature, lower target data, and the straw toughness; and generate a lower mixing instruction based on the lower mixing parameter and send the lower mixing instruction to the first mixing unit 22.

The first mixture is a mixture that enters the lower layer after mixing in the middle layer and before mixing in the lower layer of the mixer.

The mixture sampling data includes a sample particle size of the first mixture (including a percentage of particles of each size or an average size of the particles), a distribution of the sample particles, a moisture content of the sample, temperature of the sample, and a mixing degree of the sample.

In some embodiments, the processing unit may analyze the mixture sampling data based on the analyzing device 220. For example, the processing unit may obtain a sample particle size of the first mixture and a distribution of sample particles of different sizes by inspection with a laser particle size meter. As another example, the processing unit may be detected by a moisture meter, thereby obtaining a moisture content of the first mixture. As a further example, the processing unit may be detected by a temperature probe, thereby obtaining a sample temperature of the first mixture.

The middle mixing feature is a mixing feature of the mixture after being mixed by the middle mixer.

In some embodiments, the processing unit may determine the middle mixing feature based on the straw toughness, the middle mixing parameter, the middle mixer feature, the initial mixing feature, and the photosynthetic bacteria feature, by a second prediction model.

The second prediction model is a machine learning model for predicting the middle mixing feature. For example, the second prediction model may be at least one of a Neural Network (NN) model or other model. Inputs of the second prediction model include the straw toughness, the middle mixing parameter, the middle mixer feature, the initial mixing feature, and the photosynthetic bacteria feature, and outputs include the middle mixing feature.

In some embodiments, the second prediction model may be obtained by training a large number of second training samples and second labels corresponding to the second training samples. The specific training process is similar to that of the first prediction model, which will not be repeated.

Each set of the training samples in the second training samples may include a sample straw toughness, a sample middle mixing parameter, a sample middle mixer feature, a sample initial mixing feature, and a sample photosynthetic bacteria feature. The second training samples may be obtained based on historical data. The second label may be a sample middle mixing feature corresponding to the second training sample obtained by subsequent actual acquisition.

The actual mixing feature is a predicted and actual value of the mixing feature of the mixture before passing from the middle layer into the lower layer.

In some embodiments, the processing unit may construct a second feature vector based on the middle mixing feature and the mixture sampling data, look up a second vector database based on the second feature vector, and match to determine the actual mixing feature. The second vector database includes a plurality of reference actual feature vectors and a plurality of reference actual mixing feature.

The second vector database is constructed in a manner similar to that of the first vector database, as described in the preceding related description. Wherein the reference to the actual mixing feature may be obtained based on the inspection data of the plurality of locations of the mixture by the analyzing device. For example, the reference actual mixing feature may take an average of the inspection data of the plurality of locations.

The lower mixing parameter include a blade speed and a blade direction of the lower mixer.

In some embodiments, the processing unit may construct a lower feature vector based on the straw toughness, the actual mixing feature, the lower target data, the straw toughness, and the lower mixer feature; and look up a third vector database based on the lower feature vector, and match to determine the actual lower mixing parameter. The third vector database includes a plurality of sets of reference lower feature vectors and a plurality of sets of reference lower mixing parameters.

The lower mixer feature includes the number of blades of the lower mixer. The lower mixer feature may be uploaded for the user. The third vector database is constructed in a similar manner as the first vector database, as described in the preceding related description.

The lower mixer of the first mixing unit may operate based on the lower mixing parameter.

Some embodiments of the present disclosure, by determining the mixing parameters of different mixing layers of different second mixing apparatuses, the mixing process can be controlled more accurately to satisfy diversified mixing needs.

In some embodiments, the biological hydrogen-generating reactor 3 further includes a heat recovery device, and a blowing device is provided on an upper side of the conical filter 411.

The heat recovery device is used to recover the heat generated by the biological hydrogen-generating reactor 3 for fermentation of photosynthetic bacteria and provide the recovered heat to the chlorella culture unit 6. For example, the heat recovery device may be provided as a heat pump system, a plate heat exchanger, or the like.

The blowing device is used to blow away debris that the cleaning brush 424 cannot sweep away. For example, the blowing device may be provided as a centrifugal fan and/or an axial flow fan, or the like.

As shown in FIG. 13, the heat recovery device 101 and the blowing device 401 are communicatively connected to the processing unit 90.

In some embodiments, one or more blowing devices may be provided. Exemplarily, as shown in FIG. 6, a first blowing device 401-1 and a first blowing device 401-2 are provided on the upper side of the conical filter 411, the first blowing device 401-1 is provided on the inner side of the circular cover 410, and the second blowing device 401-2 is provided on the inner side wall of the solid-liquid separating cylinder 49.

In some embodiments, the heat recovery device and the blowing device are each communicatively coupled to the processing unit.

In some embodiments, the processing unit is further configured to: obtain recovered heat data of the heat recovery device; in response to determining that the recovered heat data satisfies a preset blowing condition, issue a blowing instruction to the blowing device; and determine a blow cycle of the blowing device and send the blow cycle to the blowing device.

The recovered heat data includes a recovered heat value. In some embodiments, the heat recovery device includes a heat sensor, and the recovered heat data may be measured by the heat sensor and transmitted to the processing unit 90.

The preset blowing condition is a preset condition that characterizes an imminent end of the reaction in the biological hydrogen-generating reactor 3. For example, the preset condition may be a value of recovered heat that is below a preset heat threshold.

In some embodiments, the processing unit may determine a blow cycle of the blowing device based on the conveyed mixing feature. For example, the blow cycle is negatively correlated to the average particle size of the straw. More descriptions of the conveyed mixing feature may be found below and related descriptions.

Recovering and reusing the excess heat through the heat recovery device can save energy and reduce costs. The blowing device can effectively blow away the fine particles and hard-to-clean residues adhering to the conical filter, ensure the filter surface is clean, and improve filtration effect. Additionally, removing the residue in time can prevent the filter pores from being clogged, maintain good permeability, and prolong the service life of the filter. Whether the reaction in the biological hydrogen-generating reactor 3 is about to end is determined based on the recovered heat data, and before the end, the blowing instruction is sent to turn on the blowing once to ensure that the filtering without any problem.

In some embodiments, the biological hydrogen-generating reactor 3 includes a hydrogen sensor. The hydrogen sensor is used to obtain the rate of hydrogen production of the biological hydrogen-generating reactor 3. The hydrogen sensor 330 is communicatively coupled to the processing unit as shown in FIG. 13. The hydrogen sensor may be provided in the gas collection pipe 312.

In some embodiments, the processing unit is further configured to: control the collecting device to collect a second mixture; obtain a conveyed mixing feature based on an analysis of the second mixture by the analyzing device; determine a hydrogen-producing parameter based on the conveyed mixing feature; determine a real-time hydrogen-producing parameter based on hydrogen-producing rate feature obtained by the hydrogen sensor; and generate an adjustment instruction based on the real-time hydrogen-producing parameter and adjust the hydrogen-producing parameter.

The second mixture is the mix that is delivered to the biological hydrogen-generating reactor 3 after the lower mixing is completed. The collecting device 210 may feed the collected second mixture into the analyzing device 220.

The conveyed mixing feature is an agitation feature of the lower mixer after agitation is complete. The process for determining the mixing feature based on the analyzing device 220 may be described in above descriptions.

The hydrogen-producing parameter includes a hydrogen production mixing parameter, a number of lights turned on, and brightness of the light in the biological hydrogen-generating reactor 3. The hydrogen production mixing parameter includes an agitation speed of the agitation motor 37. In some embodiments, the processing unit may determine the hydrogen-producing parameter based on the conveyed mixing feature in a variety of ways. For example, the processing unit may set the agitation speed to be higher, the number of lights turned on to be higher, the larger the particle size of the straw, the lower the concentration of photosynthesizing bacteria as well as the smaller the volume, and the lower the mixing of the straw waste with the photosynthesizing bacteria the greater the mixing speed, the greater the number of lights on and the greater the brightness.

The hydrogen-producing rate feature reflects a variation feature of the hydrogen production rate of the biological hydrogen-generating reactor 3. The processing unit may obtain the hydrogen production rate for multiple moments in a preset time period based on the hydrogen sensor and compute a statistical value. The statistical value may be an average of the hydrogen production rates for the plurality of moments.

In some embodiments, the processing unit may determine the real-time hydrogen-producing parameter based on the hydrogen-producing rate feature in a variety of ways. For example, in response to an average of the hydrogen production rate at multiple moments falling below a stage threshold, the processing unit increases the mixing speed, the number of lights on, and the brightness, and uses the adjusted mixing speed, the number of lights on, and the brightness as real-time hydrogen-producing parameter. The stage threshold may be the same or different for each time period, and the stage threshold may be preset based on experience or historical data. For example, an average or minimum of the historical hydrogen production rate for each time period in the historical data is used as the stage threshold.

Adjusting the real-time hydrogen-producing parameter according to the changes in the hydrogen production rate can improve the efficiency of the biological hydrogen-generating reactor 3.

In some embodiments, the processing unit is further configured to: determine the real-time hydrogen-producing parameter by a determination model based on a current hydrogen-producing rate feature, a historical hydrogen-producing rate feature, the photosynthetic bacteria feature, and the conveyed mixing feature.

The determination model is a machine learning model for predicting the middle mixing feature. For example, the determination model may be a Recurrent Neural Network (RNN) model or another model.

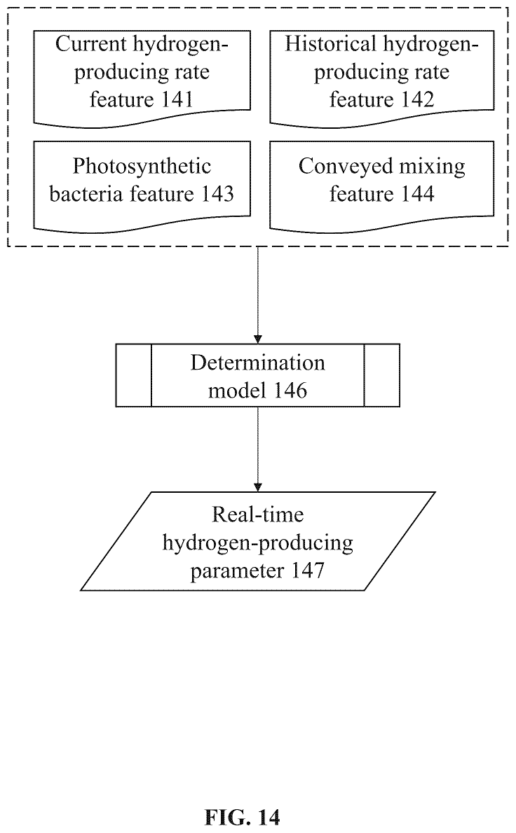

FIG. 14 is a schematic diagram illustrating a determination model according to some embodiments of the present disclosure.

As shown in FIG. 14, the inputs to the determination model 146 include the current hydrogen-producing rate feature 141, the historical hydrogen-producing rate feature 142, the photosynthetic bacteria feature 143, and the conveyed mixing feature 144, and the outputs include the real-time hydrogen-producing parameter 147. The current hydrogen-producing rate feature refers to a hydrogen-producing rate feature at the current moment. The historical hydrogen-producing rate feature refers to a hydrogen-producing rate feature in the historical data.

In some embodiments, the determination model may be obtained by training a large number of third training samples and third labels corresponding to the third training samples. The specific training process is similar to that of the first prediction model and will not be repeated.

Each set of training samples in the third training sample may include a sample hydrogen production rate, a sample hydrogen-producing rate feature, a sample photosynthetic bacteria feature, and a sample conveyed mixing feature. The third training samples may be obtained based on historical data.

The third label is an optimal hydrogen-producing parameter corresponding to the third training sample actually determined. In some embodiments, the optimal hydrogen-producing parameter may be obtained experimentally. For example, a plurality of different hydrogen-producing parameters are preset for a set of samples under a third set of training sample conditions under different experimental conditions. Under each experimental condition, changes in the rate of hydrogen production are monitored and recorded in real time. According to the experimental results, the hydrogen-producing rate feature is analyzed, and the hydrogen-producing parameter that maximizes the hydrogen production rate and maintains a relatively stable hydrogen production rate is taken as the optimal hydrogen-producing parameter. Under each experimental condition, the experiment is repeated several times, and the mean or median of the optimal hydrogen-producing parameter is taken as the third label corresponding to the third training sample.

The hydrogen production rate is not only related to the hydrogen-producing parameter, but also related to the feature of the photosynthetic bacteria, when the photosynthetic bacteria itself has reached an upper limit of the hydrogen production rate, the hydrogen-producing parameter is continued to increase, which may not increase the efficiency of the production of hydrogen, and also may be due to the increase in the number or brightness of the lamp, resulting in increased temperature, leading to bacteria inactivation, and affecting the rate of hydrogen production. Using the determination model to comprehensively analyze and determining the real-time hydrogen-producing parameter takes into account the influence of multiple factors, which improves the accuracy and reliability of the real-time hydrogen-producing parameters.

The basic concepts have been described above, and it is apparent to those skilled in the art that the foregoing detailed disclosure is intended as an example only and does not constitute a limitation of the present disclosure. While not expressly stated herein, a person skilled in the art may make various modifications, improvements, and amendments to the present disclosure. Those types of modifications, improvements, and amendments are suggested in the present disclosure, so those types of modifications, improvements, and amendments remain within the spirit and scope of the exemplary embodiments of the present disclosure.

Also, the present disclosure uses specific words to describe embodiments of the present disclosure. For example, “some embodiments” means a particular characteristic, structure, or characteristic associated with at least one embodiment of the present disclosure. Accordingly, it should be emphasized and noted that two or more references to “some embodiments” in different places in the present disclosure do not necessarily refer to the same embodiment. Additionally, certain feature, structures, or characteristics of one or more embodiments of the present disclosure may be suitably combined.

In some embodiments, the numerical parameters used in the present disclosure are approximations, which can change depending on the desired feature of individual embodiments. In some embodiments, the numerical parameters should take into account the specified number of valid digits and use a general digit retention method. While the numerical domains and parameters used to confirm the breadth of their ranges in some embodiments of the present disclosure are approximations, in specific embodiments such values are set to be as precise as possible within a feasible range.

For each of the patents, patent present disclosures, patent present disclosure disclosures, and other materials cited in the present disclosure, the entire contents of such patents, patent present disclosures, disclosures, and other materials are hereby incorporated herein by reference. It should be noted that in the event of any inconsistency or conflict between the descriptions, definitions, and/or use of terms in the materials appended to the present disclosure and those set forth herein, the descriptions, definitions and/or use of terms in the present disclosure shall control. use shall prevail.

What is claimed is:

1. A hydrogen and algal protein co-production device, comprising:
a hydrogen-producing substrate premix unit including a hydrogen-producing substrate premix box;
a biological hydrogen-generating reactor including a hydrogen-producing substrate conveying assembly, a rotary power assembly, a hollow shaft, a hydrogen-generating reaction tube, a reaction tube top cover, an agitation release assembly, and an inner wall lighting assembly, wherein a top opening of the hydrogen-generating reaction tube is equipped with the reaction tube top cover, the reaction tube top cover is rotationally connected to the hollow shaft, a top of the hollow shaft is connected to the hydrogen-producing substrate premix box through the hydrogen-producing substrate conveying assembly, a top peripheral side of the hollow shaft is connected to the rotary power assembly, a bottom of the hollow shaft is connected to the agitation release assembly, and an inner wall of the hydrogen-generating reaction tube is arranged with the inner wall lighting assembly; and
a hydrogen-producing tail liquid recovery unit being connected to the hydrogen-generating reaction tube, and the hydrogen-producing tail liquid recovery unit being connected to a hydrogen-producing tail liquid dilution unit, the hydrogen-producing tail liquid dilution unit being connected to a chlorella culture unit, and the chlorella culture unit being connected to a chlorella enrichment unit.

2. The device according to claim 1, wherein the biological hydrogen-generating reactor further includes a tapered roller bearing and a middle portion of the reaction tube top cover is rotationally connected to the hollow shaft via the tapered roller bearing.

3. The device according to claim 1, wherein
the agitation release assembly includes a plurality of horizontal pipes, a agitation vertical pipe, and a plurality of releasing ports, the plurality of horizontal pipes are connected to an outside of a bottom end of the hollow shaft, one end of each of the plurality of horizontal pipes away from the hollow shaft is fixedly connected to a top end of the agitation vertical pipe, respectively, and the plurality of releasing ports is provided at an outside of the agitation vertical pipe.

4. The device according to claim 3, wherein the plurality of releasing ports are provided at equal distances along an axial direction of the agitation vertical pipe on both sides of the agitation vertical pipe.

5. The device according to claim 1, wherein
the hydrogen-producing tail liquid recovery unit includes a heating water tank, a hydrogen-producing tail liquid conveying assembly, a solid-liquid separating mechanism, a sterilization assembly, and a sterilization box, the sterilization box is provided inside the heating water tank, and the sterilization assembly is arranged on the heating water tank, the solid-liquid separating mechanism is connected to a top of the sterilization box, a top of the solid-liquid separating mechanism extends to a top outer side of the heating water tank, and the top of the solid-liquid separating mechanism is connected to the hydrogen-generating reaction tube through the hydrogen-producing tail liquid conveying assembly.

6. The device according to claim 5, wherein
the solid-liquid separating mechanism includes a solid-liquid separating cylinder, a circular cover, a conical filter, a cleaning shaft, a shaft bracket, and a shaft-rotating power assembly, wherein a top of the sterilization box is connected to the solid-liquid separating cylinder, and a top of the solid-liquid separating cylinder is connected to a circular groove on a top of the heating water tank, the circular cover is arranged on a top of the solid-liquid separating cylinder, the shaft bracket is provided in a middle of the solid-liquid separating cylinder, the cleaning shaft being rotationally connected in the middle of the shaft bracket, a bottom end of the cleaning shaft is connected with the shaft-rotating power assembly, and a top of the cleaning shaft is connected with an inside of the conical filter.

7. The device according to claim 6, wherein a central axis of the cleaning shaft is perpendicular to a central axis of the shaft bracket, and the cleaning shaft rotates about its own central axis.

8. The device according to claim 7, wherein
the solid-liquid separating mechanism also includes a cleaning brush, a tail liquid residue outlet, a tail liquid residue box, a tail liquid backflow port, and a backflow filter, the cleaning brush is arranged on a top side of the solid-liquid separating cylinder, and bristles on a bottom side of the cleaning brush are in contact with an upper surface of the conical filter, the tail liquid residue outlet on a side of the solid-liquid separating cylinder corresponds to a position of the cleaning brush, and a bottom of the tail liquid residue outlet and a bottom of the conical filter are arranged horizontally corresponding to each other, the tail liquid residue box is fixedly connected to an outside of the solid-liquid separating cylinder, an inside of the tail liquid residue box communicates with the tail liquid residue outlet, a position of a bottom of the tail liquid residue box corresponding to a side of the solid-liquid separating cylinder is provided with the tail liquid backflow port, the tail liquid backflow port is connected to an interior of the tail liquid residue box, and the backflow filter is arranged in the tail liquid backflow port.

9. The device according to claim 5, wherein
the hydrogen-producing tail liquid dilution unit includes a first delivery pipe, a first constant-flow pump, a dilution box, and a dilution addition assembly, a bottom side of the sterilization box is connected to one end of the first delivery pipe, the other end of the first delivery pipe passes through a side of the heating water tank and is connected to an inlet of the first constant-flow pump, an outlet of the first constant-flow pump is connected to the dilution box, and the dilution box is arranged with the dilution addition assembly.

10. The device according to claim 9, wherein
the chlorella culture unit includes a diluent conveying assembly, a chlorella culture tank, an aeration assembly, a raw liquid addition assembly, and a chlorella lighting assembly, the dilution box is connected to the chlorella culture tank via the diluent conveying assembly, the chlorella culture tank is provided with the aeration assembly and the chlorella lighting assembly, respectively, and the raw liquid addition assembly is arranged on a top of the chlorella culture tank.

11. The device according to claim 10, wherein
the chlorella culture unit further includes a chlorella output assembly and a first spiral transparent pipe, the chlorella culture tank is connected to a top end of the first spiral transparent pipe via the chlorella output assembly, and the first spiral transparent pipe is wrapped around a light mixing utilization unit.

12. The device according to claim 11, wherein
the chlorella enrichment unit includes a waste liquid inflow recovery chamber, a waste liquid delivery pipe, and an enrichment assembly, a top of the waste liquid inflow recovery chamber is arranged with the enrichment assembly, and the enrichment assembly is connected to a bottom of the first spiral transparent pipe via the waste liquid delivery pipe.

13. The device according to claim 12, wherein
the enrichment assembly includes an enrichment mounting sleeve, a dismantling sleeve, an enrichment round cover, and an enrichment filter, the enrichment mounting sleeve is interspersed at a top of the waste liquid inflow recovery chamber, a top of the enrichment mounting sleeve is snapped with a bottom of the dismantling sleeve, the enrichment filter is provided at a bottom of the dismantling sleeve, a top end of the dismantling sleeve is snap with a bottom of the enrichment round cover, and a middle portion of the enrichment round cover is connected to the waste liquid delivery pipe.

14. The device according to claim 13, wherein further includes a processing unit, the hydrogen-producing substrate premix unit includes a first mixing unit, and the first mixing unit includes a multi-layer mixer;
the processing unit is configured as:
determining a mixing parameter of the first mixing unit based on a straw initial feature.

15. The device according to claim 14, wherein the processing unit is further configured to:
determine mixing target data based on straw types and moisture content;
determine an upper mixing parameter based on the straw initial feature and a photosynthetic bacteria feature;
determine an initial mixing feature based on the upper mixing parameter, an upper mixer feature, the straw initial feature, the photosynthetic bacteria feature, and an initial mixing degree; and
determine a middle mixing parameter based on the initial mixing feature, a middle mixer feature, the mixing target data, and straw toughness.

16. The device according to claim 15, wherein further includes a collecting device and an analyzing device; and a mixture sampling port is arranged with a bottom of the hydrogen-producing substrate premix unit;
the processing unit is further configured to:
control the collecting device to obtain a first mixture from the mixture sampling port and feed the first mixture to the analyzing device for analysis to obtain mix sampling data;
determine a middle mixing feature based on the straw toughness, the middle mixing parameter, the middle mixer feature, the initial mixing feature, and the photosynthetic bacteria feature;
determine an actual mixing feature based on the middle mixing feature and the mix sampling data;
determine a lower mixing parameter based on the actual mixing feature, a lower mixer feature, lower target data, and the straw toughness; and
generate a lower mixing instruction based on the lower mixing parameter and send the lower mixing instruction to the first mixing unit.

17. The device according to claim 14, wherein the biological hydrogen-generating reactor further includes a heat recovery device, and a blowing device is provided on an upper side of the conical filter;
the processing unit is further configured to:
obtain recovered heat data of the heat recovery device;
in response to determining that the recovered heat data satisfies a preset blowing condition, issue a blowing instruction to the blowing device; and
determine a blow cycle of the blowing device and send the blow cycle to the blowing device.

18. The device according to claim 16, wherein the biological hydrogen-generating reactor includes a hydrogen sensor;
the processing unit is further configured to:
control the collecting device to collect a second mixture;
obtain a conveyed mixing feature based on an analysis of the second mixture by the analyzing device;
determine a hydrogen-producing parameter based on the conveyed mixing feature;
determine a real-time hydrogen-producing parameter based on a hydrogen-producing rate feature obtained by the hydrogen sensor; and
generate an adjustment instruction based on the real-time hydrogen-producing parameter and adjust the hydrogen-producing parameter.

19. The device according to claim 18, wherein the processing unit is further configured to:
determine the real-time hydrogen-producing parameter by a determination model based on a current hydrogen-producing rate feature, a historical hydrogen-producing rate feature, the photosynthetic bacteria feature, and the conveyed mixing feature.
